(19) United States
(12) Patent Application Publication
(10) Pub. No.: US 2003/0092156 A1
(43) Pub. Date: May 15, 2003

(54) Antibodies specifically binding cyclic nucleotide PDEs

(76) Inventors: Stephen C. Phillips, Wingham (GB); Ian Harrow, Herne Bay (GB); Jerry Lanfear, Great Chart (GB); Lindsay Fawcett, Canterbury (GB); Olga Bandman, Mountain View, CA (US)

Correspondence Address: INCYTE GENOMICS, INC., 3160 PORTER DRIVE, PALO ALTO, CA, 94304, US

(21) Appl. No.: 10/094,168

(22) Filed: Mar. 7, 2002

Related U.S. Application Data
(63) Continuation-in-part of application No. 09/595,514, filed on Jun. 14, 2000, now Pat. No. 6,416,991.
Application No. 09/595,514 is a continuation-in-part of application No. 09/226,741, filed on Jan. 7, 1999, now Pat. No. 6,100,037.

(51) Int. Cl. G01N 33/53; C12N 9/16; C07K 16/40

(52) U.S. Cl. 435/196; 530/388.26; 435/7.1

(57) ABSTRACT

The invention provides human cyclic nucleotide phosphodiesterases (HSPDE10A), polynucleotides that encode HSPDE10A, and antibodies that specifically bind HSPDE10A. The invention also provides expression vectors, host cells, agonists, and antagonists and methods for diagnosing or treating disorders associated with expression of HSPDE10A.

This application is a continuation-in-part of U.S. Ser. No. 09/595,514, filed Jun. 14, 2000, which was a continuation-in-part of U.S. Pat. No. 6,100,037, which matured from U.S. Ser. No. 09/226,741, filed Jan. 7, 1999.

### FIELD OF THE INVENTION

This invention relates to human cyclic nucleotide phosphodiesterases, polynucleotides which encode these phosphodiesterases and antibodies that specifically bind the phophodiesterases, and their mammalian variants and to their use to diagnose, to stage, to treat, or to monitor the progression or treatment of cancer and immune disorders.

### BACKGROUND OF THE INVENTION

Cyclic nucleotides (cAMP and cGMP) function as intracellular second messengers to transduce a variety of extracellular signals including hormones, light, and neurotransmitters. Cyclic nucleotide phosphodiesterases (PDEs) degrade cyclic nucleotides to the corresponding monophosphates, thereby regulating the intracellular concentrations of cyclic nucleotides and their effects on signal transduction. At least seven families of mammalian PDEs have been identified based on substrate specificity and affinity, and sensitivity to cofactors and inhibitory drugs (Beavo (1995) Physiol Rev 75:725-748). Several of these families contain distinct genes, many of which are expressed in different tissues as splice variants. Within families, there are multiple isozymes and multiple splice variants of those isozymes. The existence of multiple PDE families, isozymes, and splice variants presents an opportunity for regulation of cyclic nucleotide levels and functions.

Type 1 PDEs (PDE1s) are Ca²⁺/calmodulin-dependent and appear to be encoded by three different genes, each having at least two different splice variants. PDEls have been found in the lung, heart, and brain. Some of the Ca²⁺/calmodulin-dependent PDEs are regulated in vitro by phosphorylation and dephosphorylation. Phosphorylation of PDE1 decreases the affinity of the enzyme for calmodulin, decreases PDE activity, and increases steady state levels of cAMP. PDE2s are cGMP stimulated PDEs that are localized in the brain and are thought to mediate the effects of cAMP on catecholamine secretion. PDE3s are one of the major families of PDEs present in vascular smooth muscle. PDE3s are inhibited by cGMP, have high specificity for cAMP as a substrate, and play a role in cardiac function. One isozyme of PDE3 is regulated by one or more insulin-dependent kinases. PDE4s are the predominant isoenzymes in most inflammatory cells, and some PDE4s are activated by cAMP-dependent phosphorylation. PDE5s are thought to be cGMP specific but may also hydrolyze cAMP. High levels of PDE5s are found in most smooth muscle preparations, in platelets, and in the kidney. PDE6s play a role in vision and are regulated by light and cGMP. The PDE7 class, consisting of only one known member, is cAMP-specific and is most closely related to PDE4. PDE7 is not inhibited by rolipram, a specific inhibitor of PDE4 (Beavo, supra). PDE8 and PDE9 represent two new families of PDEs. PDE8s are cAMP specific, most closely related to PDE4, insensitive to rolipram, and sensitive to dipyridimole. PDE9s are cGMP specific and sensitive only to the PDE inhibitor, zaprinast.

PDEs are composed of a catalytic domain of about 270 amino acids, an N-terminal regulatory domain responsible for binding cofactors, and, in some cases, a C-terminal domain of unknown function. A conserved motif, HDXXHXGXXN, has been identified in the catalytic domain of all PDEs. In PDE5, an N-terminal cGMP binding domain spans about 380 amino acid residues and comprises tandem repeats of the conserved sequence motif N(R/K)XnFX₃DE (McAllister-Lucas et al. (1993) J Biol Chem 268:22863-22873). The NKXnD motif has been shown by mutagenesis to be important for cGMP binding (Turko et al. (1996) J Biol Chem 271:22240-22244). PDE families display approximately 30% amino acid identity within the catalytic domain; however, isozymes within the same family typically display about 85-95% identity in this region (e.g. PDE4A vs PDE4B). Furthermore, within a family there is extensive sequence similarity (>60%) outside the catalytic domain; while across families, there is little or no sequence similarity.

Many functions of immune and inflammatory responses are inhibited by agents that increase intracellular levels of cAMP (Verghese et al. (1995) Mol Pharmacol 47:1164-1171). A variety of diseases have been attributed to increased PDE activity and associated with decreased levels of cyclic nucleotides. A form of diabetes insipidus in the mouse has been associated with increased PDE4 activity, and an increase in low-Kₘ cAMP PDE activity has been reported in leukocytes of atopic patients. Defects in PDEs have also been associated with retinal disease. Retinal degeneration in the rd mouse, autosomal recessive retinitis pigmentosa in humans, and rod/cone dysplasia 1 in Irish Setter dogs have been attributed to mutations in the PDE6B gene. PDE3 has been associated with cardiac disease.

Many inhibitors of PDEs have been identified and have undergone clinical evaluation. PDE3 inhibitors are being developed as antithrombotic agents, antihypertensive agents, and as cardiotonic agents useful in the treatment of congestive heart failure. Rolipram, a PDE4 inhibitor, has been used in the treatment of depression, and other inhibitors of PDE4 are undergoing evaluation as anti-inflammatory agents. Rolipram has also been shown to inhibit lipopolysaccharide induced TNF-α which has been shown to enhance HIV-1 replication in vitro. Therefore, rolipram may inhibit HIV-1 replication (Angel et al. (1995) AIDS 9:1137-44). Additionally, rolipram, based on its ability to suppress the production of cytokines, such as TNF-α and β and interferon γ, has been shown to be effective in the treatment of encephalomyelitis. Rolipram may also be effective in treating tardive dyskinesia and was effective in treating multiple sclerosis in an experimental animal model (Sommer et al. (1995) Nature Med 1:244-248; Sasaki et al. (1995) Eur J Pharmacol 282:71-76).

Theophylline is a nonspecific PDE inhibitor used in the treatment of bronchial asthma and other respiratory diseases. Theophylline is believed to act on airway smooth muscle function and in an anti-inflammatory or immunomodulatory capacity in the treatment of respiratory diseases (Banner and Page (1995) Eur Respir J 8:996-1000). Pentoxifylline is another nonspecific PDE inhibitor used in the treatment of intermittent claudication and diabetes-induced peripheral vascular disease. Pentoxifylline is also known to block TNF-α production and may inhibit HIV-1 replication (Angel, supra).

PDEs have also been reported to effect cellular proliferation of a variety of cell types and have been implicated in various cancers. Bang et al. (1994; Proc Natl Acad Sci 91:5330-5334) reported that growth of prostate carcinoma cell lines DU 145 and LNCaP was inhibited by delivery of cAMP derivatives and phosphodiesterase inhibitors. These cells also showed a permanent conversion in phenotype from epithelial to neuronal morphology. Others have suggested that PDE inhibitors have the potential to regulate mesangial cell proliferation and lymphocyte proliferation (Matousovic et al. (1995) J Clin Invest 96:401-410; Joulain et al. (1995) J Lipid Mediat Cell Signal 11:63-79, respectively). Finally, Deonarain et al.(1994; Br J Cancer 70:786-94) describe a cancer treatment that involves intracellular delivery of phosphodiesterases to particular cellular compartments of tumors which results in cell death.

The discovery of new human cyclic nucleotide phosphodiesterases, polynucleotides that encode these phosphodiesterases and antibodies that specifically bind the phophodiesterases satisfies a need in the art by providing new compositions which are useful to diagnose, to stage, to treat, or to monitor the progression or treatment of cancer and immune disorders.

### SUMMARY OF THE INVENTION

The invention is based on the discovery of new human cyclic nucleotide phosphodiesterases referred to collectively as “HSPDE10A” and individually as “HSPDE10A1” and “HSPDE10A2”, the polynucleotides encoding HSPDE10A, and antibodies that specifically bind HSPDE10OA, and on the use of these compositions to diagnose, to stage, to treat, or to monitor the progression or treatment of cancer and immune disorders.

The invention features a purified protein comprising the amino acid sequence of SEQ ID NO:1, SEQ ID NO:3, or portions thereof. The invention provides a purified variant having at least 90% amino acid sequence identity to the amino acid sequence of SEQ ID NO:1, SEQ ID NO:3, or portions thereof.

The invention provides an isolated polynucleotide encoding the protein comprising the amino acid sequence of SEQ ID NO:1, SEQ ID NO:3, or portions thereof. The invention also provides an isolated polynucleotide which hybridizes under stringent conditions to the polynucleotide encoding the protein comprising the amino acid sequence of SEQ ID NO:1, SEQ ID NO:3, or portions thereof. The invention further provides an isolated polynucleotide having a sequence which is complementary to the polynucleotide encoding the protein comprising the amino acid sequence of SEQ ID NO:1, SEQ ID NO:3, or portions thereof. The invention still further provides an isolated polynucleotide comprising the nucleic acid sequence of SEQ ID NO:2, SEQ ID NO:4, or fragments thereof and isolated variants having at least 70% polynucleotide sequence identity to the polynucleotide comprising the polynucleotide sequence of SEQ ID NO:2, SEQ ID NO:4, or fragments thereof. The invention yet still further provides an isolated polynucleotide having a sequence complementary to the polynucleotide comprising the polynucleotide sequence of SEQ ID NO:2, SEQ ID NO:4, or fragments thereof.

The invention provides a method for detecting a polynucleotide in a sample containing nucleic acids comprising hybridizing the complement of the polynucleotide to at least one of the nucleic acids of the sample, thereby forming a hybridization complex, and detecting the hybridization complex, wherein the presence of the hybridization complex indicates the presence of the polynucleotide in the sample. In one aspect, the method further comprises amplifying the nucleic acids of the sample prior to hybridization. The invention also provides an expression vector containing the polynucleotide encoding the protein comprising the amino acid sequence of SEQ ID NO:1 or SEQ ID NO:3. In one aspect, the expression vector is contained within a host cell. The invention further provides a method for using a polynucleotide to produce a protein comprising culturing the host cell under conditions for expression of the protein and recovering the protein from the culture. In one embodiment, the method produces a purified protein having the amino acid sequence of SEQ ID NO:1, SEQ ID NO:3, or portions thereof.

The invention provides a method for using the protein to screen a plurality of molecules or compounds to identify a ligand comprising combining the protein with the molecules or compounds under conditions to allow specific binding and detecting specific binding, thereby identifying a ligand that specifically binds the protein. In one aspect, the molecules and compounds are selected from the group consisting of DNA molecules, RNA molecules, peptide nucleic acid molecules, peptides, proteins, agonists, antagonists, inhibitors, drug compounds and pharmaceutical agents.

The invention provides a purified antibody that specifically binds the protein comprising the amino acid sequence of SEQ ID NO:1 or SEQ ID NO:3. The invention also provides a method for using a protein or a portion thereof to screen a plurality of antibodies to identify an antibody that specifically binds the protein comprising contacting a plurality of antibodies with the protein under conditions to form an antibody:protein complex, and dissociating the antibody from the antibody:protein complex, thereby obtaining antibody that specifically binds the protein. In one aspect, the antibody is selected from a polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein.

The invention provides methods for using a protein to prepare and purify polyclonal and monoclonal antibodies that specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining polyclonal antibodies that specifically bind the protein. The invention also provides a polyclonal antibody that specifically binds HSPDE10A. The method for preparing a monoclonal antibody comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture. The invention further provides a monoclonal antibody that specifically binds HSPDE10A.

The invention provides a method for using an antibody that specifically binds HSPDE10A to detect expression of a protein in a sample, the method comprising combining the antibody with a sample under conditions for formation of antibody:protein complexes; and detecting complex formation, wherein complex formation indicates expression of the protein HSPDE10A in the sample. In one aspect, the sample is biopsied tissue. In another aspect, the amount of complex formation when compared to standards is diagnostic of a cancer and immune disorder. In a further aspect, the antibody is attached to a substrate. The invention also provides a method for using the antibody that specifically binds the protein in an assay to evaluate treatment of adenofibromatous hyperplasia of the prostate comprising contacting the antibody that specifically binds HSPDE10A with a sample from a patient, detecting complex formation between the antibody and protein, comparing complex formation with standards, wherein the difference in complex formation indicates the efficacy of treatment. In one aspect, the antibody is attached to a substrate.

The invention provides a method for immunopurification of a protein comprising attaching an antibody to a substrate, exposing the antibody to a sample containing protein under conditions to allow antibody:protein complexes to form, dissociating the protein from the complex, and collecting purified protein.

The invention provides compositions comprising isolated polynucleotides which encode HSPDE10A, purified HSPDE10A, and isolated antibodies, agonists or antagonists that specifically bind HSPDE10A.

The invention provides a method for treating adenofibromatous hyperplasia of the prostate as it is associated with decreased expression or activity of HSPDE10A comprising administering to a subject in need of such treatment a composition comprising a purified protein having the amino acid sequence of SEQ ID NO:1, SEQ ID NO:3, or portions thereof, in conjunction with a pharmaceutical carrier. The invention also provides a method for treating adenofibromatous hyperplasia of the prostate comprising administering to a subject in need of such treatment a composition comprising an agonist that specifically binds the protein having the amino acid sequence of SEQ ID NO:1, SEQ ID NO:3, or portions thereof. The invention further provides a method for treating a disorder associated with increased expression or activity of HSPDE10A, the method comprising administering to a subject in need of such treatment an antagonist of the protein having the amino acid sequence of SEQ ID NO:1, SEQ ID NO:3, or portions thereof.

### BRIEF DESCRIPTION OF THE FIGURES AND TABLE

FIGS. 1A, 1B, 1C, 1D, and 1E show the amino acid sequence (SEQ ID NO:1) and nucleic acid sequence (SEQ ID NO:2) of HSPDE10A1. The alignment was produced using MACDNASIS PRO software (Hitachi Software Engineering, South San Francisco Calif.).

FIGS. 2A, 2B, 2C, 2D, 2E, and 2F show the amino acid sequence (SEQ ID NO:3) and nucleic acid sequence (SEQ ID NO:4) of HSPDE10A2. The alignment was produced using MACDNASIS PRO software.

FIGS. 3A, 3B, 3C, 3D, and 3E show the amino acid sequence alignments between HSPDE10A1 (SEQ ID NO:1), HSPDE10A2 (SEQ ID NO:3), and human PDE5, HPDE5A1 (g3355606; SEQ ID NO:5), produced using the MEGALIGN program of LASERGENE software (DNASTAR, Madison Wis.).

FIGS. 4A and 4B show the activity assay for HSPDE10A1 using cAMP and cGMP as substrates, respectively. The positive X axis represents the substrate concentration (mM), and the positive Y axis represents the reaction velocity in pmoles/minute/ml enzyme. Kₘ and Vₘₐₓ values for the enzyme activity with each substrate were calculated from a Michaelis-Menten plot using the “Fit Curve” Microsoft Excel extension program.

FIGS. 5A and 5B show the membrane-based northern analysis of HSPDE10A expression in human tissues. The X axis presents the various tissues analyzed, and the Y axis presents various size markers. The arrow indicates the location of the major (˜7.5 kb) transcript of HSPDE10A.

FIG. 6 shows the expression of full length HSPDE10A1 in Sf9 cells (arrow; predicted molecular weight ˜56 kDa). Lane 1 shows various size markers and their molecular weights. Lanes 2 and 4, infected cells at 64,000 and 12,800 cell equivalents, respectively, show HSPDE10A1. Lanes 3 and 5, mock-infected cells at 64,000 and 12,800 cell equivalents, respectively, fail to show the presence of HSPDE10A1.

FIG. 7 shows the northern analysis for HSPDE10A1 produced using the LIFESEQ Gold database (Incyte Genomics, Palo Alto Calif.). In the first range, the first column presents the tissue categories; the second column, the number of clones in the tissue category; the third column, the number of libraries in which at least one transcript was found; the fourth column, absolute abundance of the transcript; and the fifth column, percent abundance of the trancript. In the second range, the first column lists the library name, the second column, the number of clones sequenced for that library; the third column, description of the tissue; the fourth column, absolute abundance of the transcript; and the fifth column, percent abundance of the trancript.

Table 1 shows the effects of various PDE inhibitors on the activity of HSPDE10A1. Assays were carried out using cGMP as a substrate at a concentration of 0.17 mM, equal to ˜⅓ of the Kₘ of cGMP. Inhibitors were tested over a range of concentrations from ˜0.5 to ˜110 mM. IC₅₀ (or Kᵢ) values were extrapolated from the dose response curves.

Table 2 shows the programs, their descriptions, references, and threshold parameters used to analyze HSPDE10A.

### DESCRIPTION OF THE INVENTION

Before the present proteins, polynucleotides, and methods are described, it is understood that this invention is not limited to the particular machines, materials and methods described as these may vary. It is also to be understood that the terminology used herein is for the purpose of describing particular embodiments only and is not intended to limit the scope of the present invention which will be limited only by the appended claims.

It must be noted that as used herein and in the appended claims, the singular forms “a”, “an”, and “the” include plural reference unless the context clearly dictates otherwise. For example, a reference to “a host cell” includes a plurality of such host cells, and a reference to “an antibody” is a reference to one or more antibodies and equivalents thereof known to those of skill in the art.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All patents and publications mentioned herein are hereby incorporated by reference and have been cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“HSPDE10A” refers to the amino acid sequences of a purified HSPDE10A obtained from any species including bovine, ovine, porcine, murine, equine, rodent, and preferably the human species, from any source, whether natural, synthetic, semi-synthetic, or recombinant.

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an immunogenic epitope, structural feature, or region of an oligopeptide, peptide, or protein which is capable of inducing formation of an antibody that specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody of diagnostic or therapeutic interest. The arrangement of two to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

The “complement” of a polynucleotide of the Sequence Listing refers to a nucleic acid molecule which is completely complementary over its full length and which will hybridize to the polynucleotide or an mRNA under conditions of high stringency.

“Derivative” refers to a polynucleotide or a protein that has been subjected to a chemical modification. Derivatization of a polynucleotide can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or upregulated or a decreased or downregulated expression as detected by absence, presence, or at least two-fold change in the amount of transcribed messenger RNA or translated protein in a sample.

An “expression profile” is a representation of gene expression in a sample. A nucleic acid expression profile is produced using sequencing, hybridization, or amplification technologies and mRNAs or cDNAs from a sample. A protein expression profile mirrors the nucleic acid expression profile and uses labeling moieties or antibodies to quantify the protein expression in a sample. The nucleic acids, proteins, or antibodies may be used in solution or attached to a substrate, and their detection is based on methods and labeling moieties well known in the art.

“Disorder” refers to conditions, diseases or syndromes in which the polynucleotides and HSPDE10A are differentially expressed such as adenofibromatous hyperplasia of the prostate.

“Fragment” refers to a chain of consecutive nucleotides from about 61 to about 5000 base pairs in length. Fragments may be used in PCR or hybridization technologies to identify related nucleic acid molecules and in binding assays to screen for a ligand. Nucleic acids and their ligands identified in this manner are useful as therapeutics to regulate replication, transcription or translation.

A “hybridization complex” is formed between a polynucleotide and a nucleic acid of a sample when the purines of one molecule hydrogen bond with the pyrimidines of the complementary molecule, e.g., 5′-A-G-T-C-3′ base pairs with 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997) Nucleic Acids Res 25:3389-3402. BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues. In proteins, similarity exceeds identity in that conservative substitutions, for example, valine for leucine or isoleucine, are counted in calculating the reported percentage. Substitutions which are considered to be conservative are well known in the art.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a cDNA or protein. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a complementary site on a cDNA molecule or polynucleotide, or to an epitope or a protein. Such ligands stabilize or modulate the activity of polynucleotides or proteins and may be composed of inorganic or organic substances including nucleic acids, proteins, carbohydrates, fats, and lipids.

“Oligonucleotide” refers a single stranded molecule from about 18 to about 60 nucleotides in length which may be used in hybridization or amplification technologies or in regulation of replication, transcription or translation. Equivalent terms are amplimer, primer, and oligomer.

“Polynucleotide” refers to an isolated cDNA, nucleic acid molecule, or any fragment or complement thereof. It may have originated recombinantly or synthetically, be double-stranded or single-stranded, represent coding and/or noncoding sequence, an exon with or without an intron from a genomic DNA molecule.

The phrase “polynucleotide encoding a protein” refers to a nucleic acid sequence that closely aligns with sequences which encode conserved regions, motifs or domains that were identified by employing analyses well known in the art. These analyses include BLAST (Basic Local Alignment Search Tool; Altschul (1993) J Mol Evol 36: 290-300; Altschul et al. (1990) J Mol Biol 215:403-410) which provides identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, p. 6076, column 2).

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a polynucleotide that hybridizes to at least one nucleic acid in a sample. Where targets are single stranded, probes are complementary single strands. Probes can be labeled with reporter molecules for use in hybridization reactions including Southern, northern, in situ, dot blot, array, and like technologies or in screening assays.

“Protein” refers to a polypeptide. peptide, or any portion thereof. A “portion” of a protein refers to that length of amino acid sequence which would retain at least one biological activity, a domain identified by PFAM or PRINTS analysis or an antigenic epitope of the protein identified using Kyte-Doolittle algorithms of the PROTEAN program (DNASTAR). An “oligopeptide” is an amino acid sequence from about five residues to about 15 residues that is used as part of a fusion protein to produce an antibody.

“Purified” refers to any molecule or compound that is separated from its natural environment and is from about 60% free to about 90% free from other components with which it is naturally associated.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue; a tissue print; a fingerprint, buccal cells, skin, or hair; and the like.

“Specific binding” refers to a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

“Substrate” refers to any rigid or semi-rigid support to which polynucleotides or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The invention is based on the discovery of new human cyclic nucleotide phosphodiesterases (HSPDE10A), the polynucleotides encoding HSPDE10A, and antibodies that specifically bind the phosphodiesterases, and on the use of these compositions to diagnose, to stage, to treat, or to monitor the progression or treatment of cancer and immune disorders.

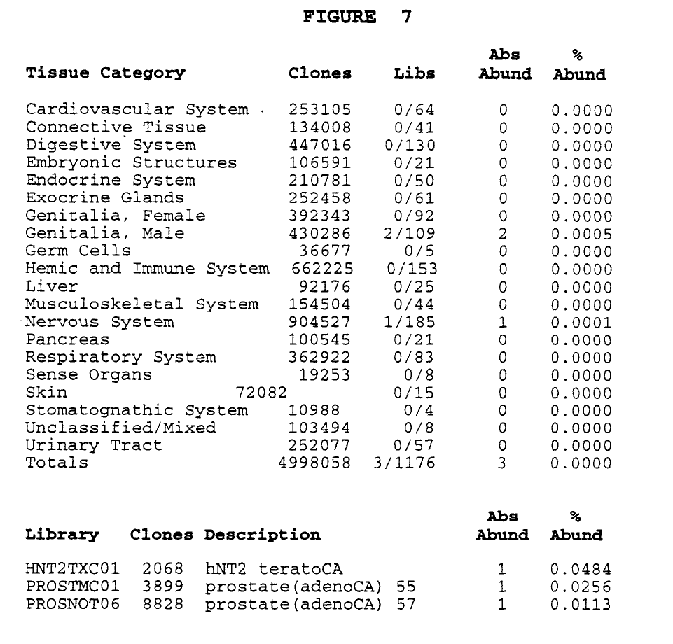

Nucleic acids encoding the HSPDE10A of the present invention were identified in Incyte Clone 826776 from the prostate cDNA library (PROSTUT04) using BLAST analysis and human PDE5 (GI 3355606) as a query sequence against the LIFESEQ database (Incyte Genomics, Palo Alto Calif.). Full length cDNA sequences of HSPDE10A were obtained from a human skeletal muscle library using the complete cDNA insert of Incyte Clone 826776 as a hybridization probe. Clone 826776 has identity with the nucleotide sequence encoding HSPDE10A from nucleotide 627 to nucleotide 888. The oligonucleotide of SEQ ID NO:2 from about nucleotide 1168 to about nucleotide 1212 is useful in hybridization or amplification technologies to identify SEQ ID NO:2 and to distinguish between SEQ ID NO:2 and a related sequence. The oligonucleotide of SEQ ID NO:4 from about nucleotide 1183 to about nucleotide 1227 is useful in hybridization or amplification technologies to identify SEQ ID NO:4 and to distinguish between SEQ ID NO:4 and a related sequence. FIG. 7 shows the electronic northern analysis for HSPDE10A.

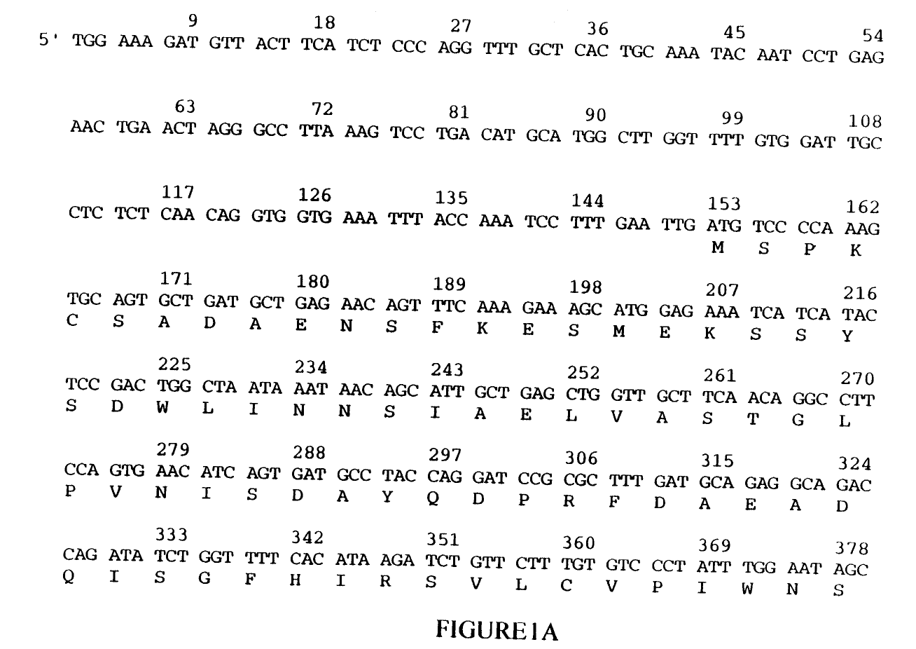

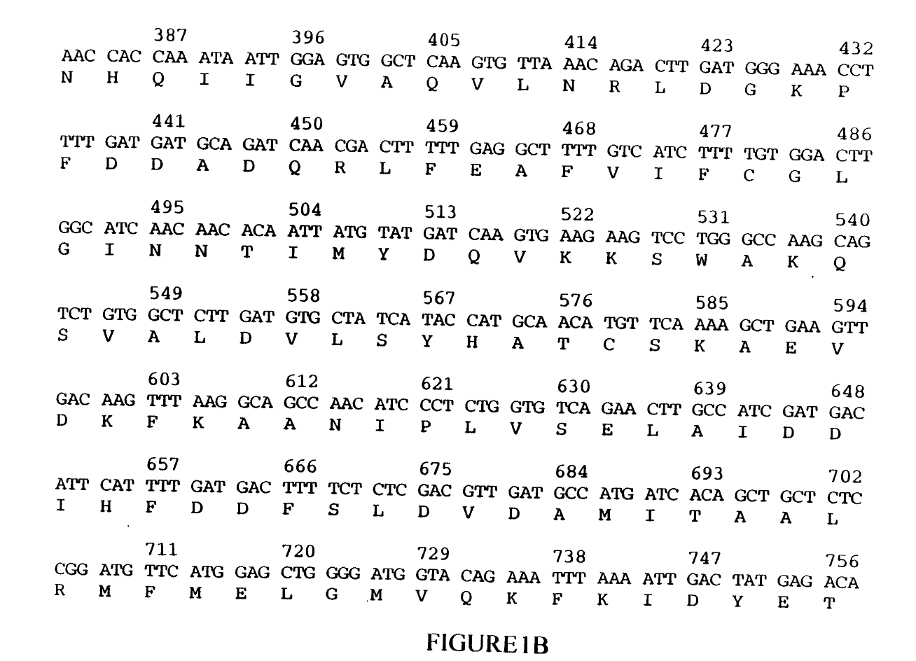

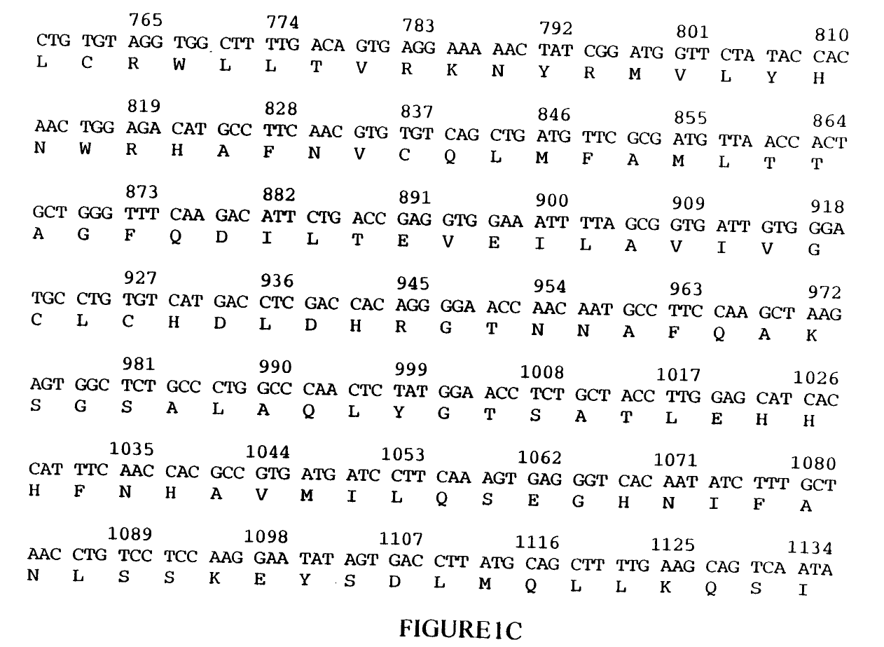

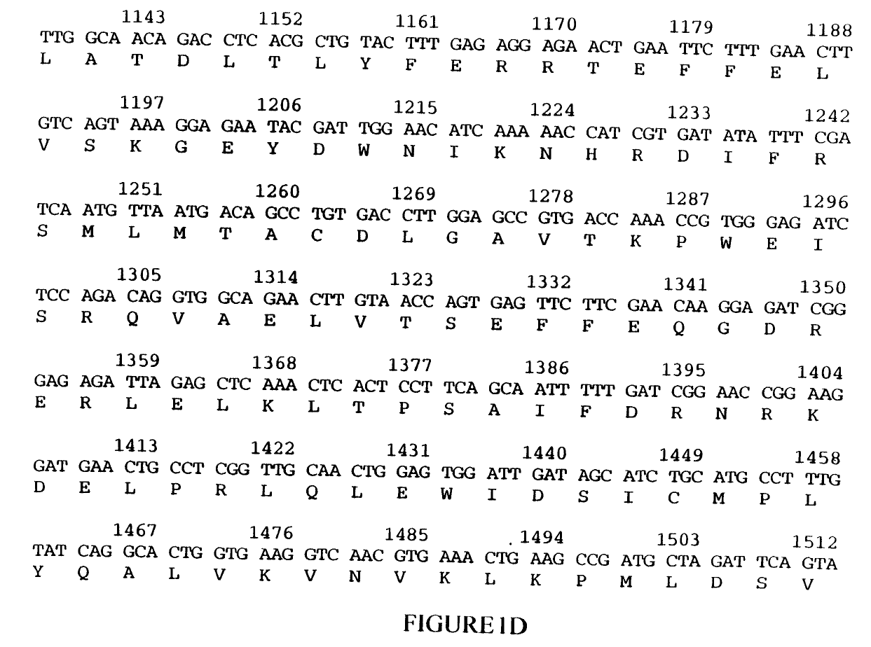

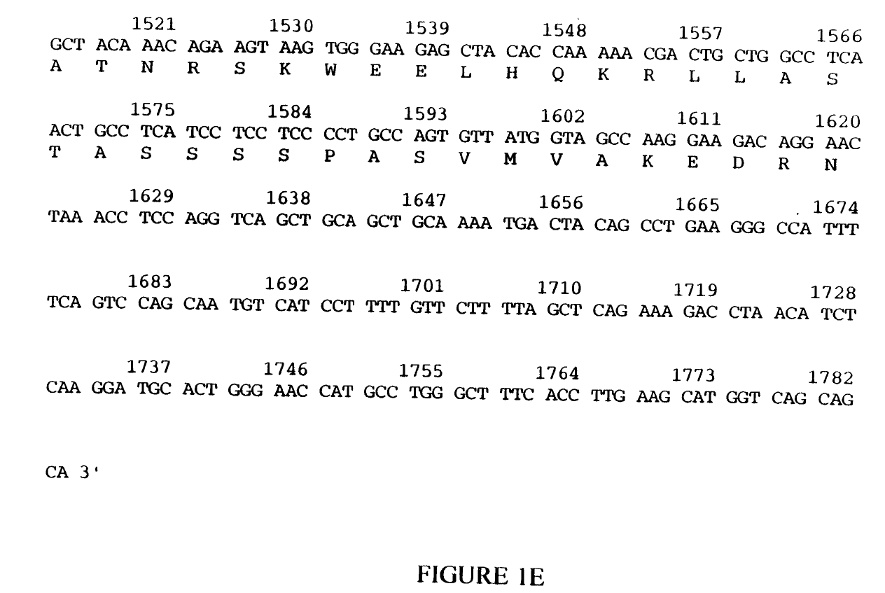

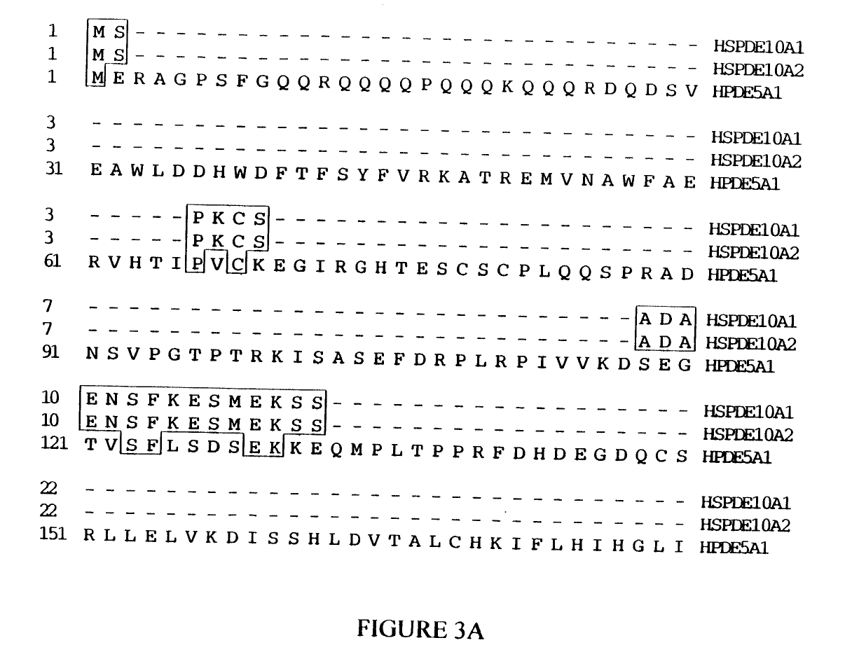

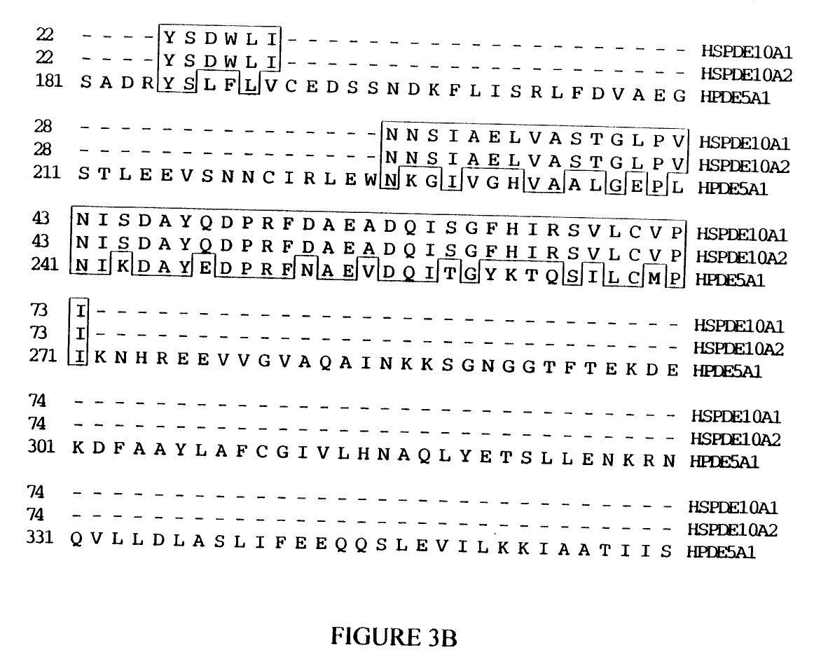

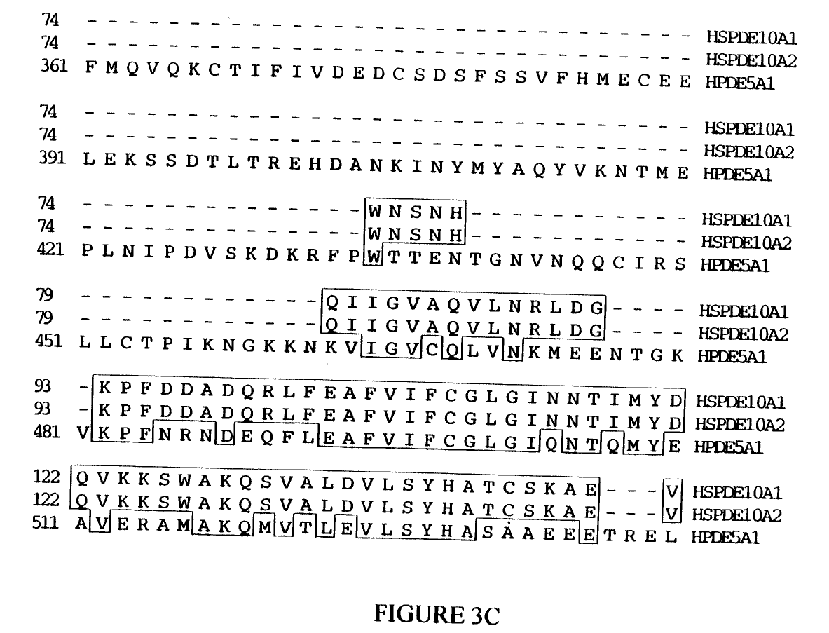

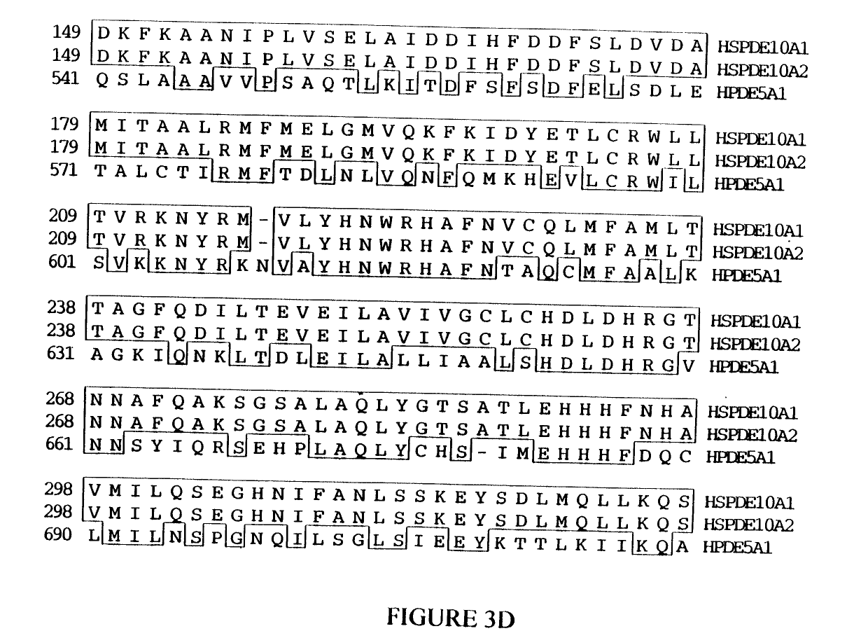

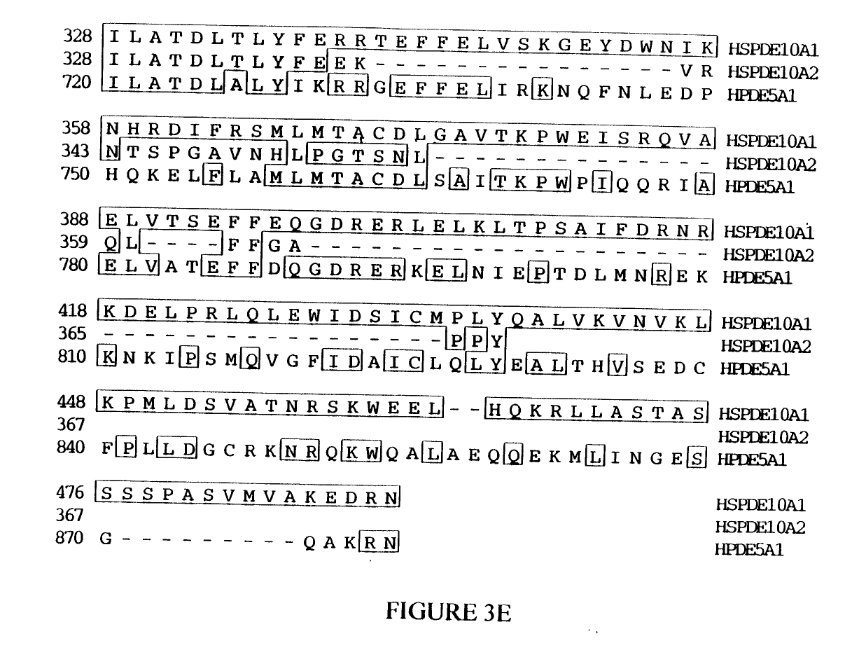

In one embodiment, the invention encompasses a protein comprising the amino acid sequence of SEQ ID NO:1, as shown in FIGS. 1A-1E. HSPDE10A1 is 490 amino acids in length and has a putative cGMP binding motif in the sequence N₈₈RLDGKPFDDAD of SEQ ID NO:1 and a PDE signature motif at H₂₆₀DLDHRGTNN of SEQ ID NO:1. As shown in FIGS. 3A-3E, HSPDE10A1 has chemical and structural similarity with human PDE5, HSPDE5A1 (g3355606; SEQ ID NO:5). In particular, HSPDE10A1 and HSPDE5A1 share 42% identity. The ˜270 amino acid catalytic domain found in all PDEs extends between residues F₁₉₆ and R₄₅₈ in HSPDE10A1 and is 50% identical to HSPDE5A1 in this region. The putative cGMP binding motif in HSPDE10A1 beginning at residue N₈₈ is coincident with the tandem repeat motif for cGMP binding in HSPDE5A1 beginning at residue N₄₇₂, and the PDE signature sequence for HSPDE10A1 beginning at residue H₂₆₀ is conserved in HSPDE5A as well. HSPDE10A1 shares a slightly lesser degree of homology, ranging from 25% to 44%, with other representatives of PDE families 1, 2, 3, 4, 6, 7, 8, and 9 (data not shown). Portions of HSPDE10A which are hydrophillic and appropriate to use as antigenic epitopes include residues 10-25, 45-60, 88-103, 145-158, and 260-275. Antibodies produced using antigenic epitopes comprising residues 350-365 and 396-411 would distinguish HSPDE10A1 from HSPDE10A2. These epitopes are synthesized, coupled to keyhole limpet hemocyanin (KLH, Sigma-Aldrich, St. Louis Mo.) and used to produce antibodies which bind specifically to HSPDE10A and are diagnostics for examining biopsied prostate tissues potentially affected by adenofibromatous hyperplasia.

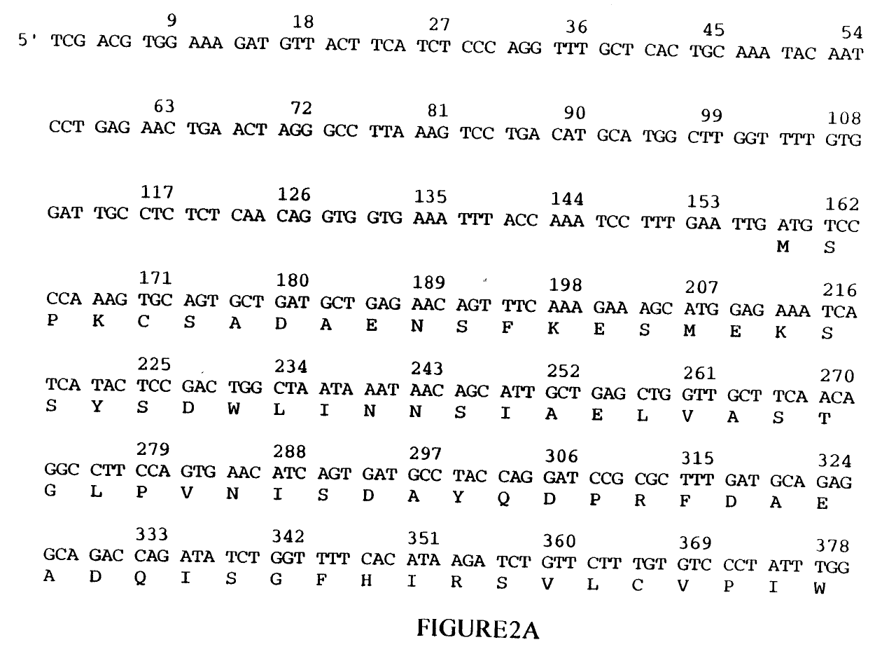

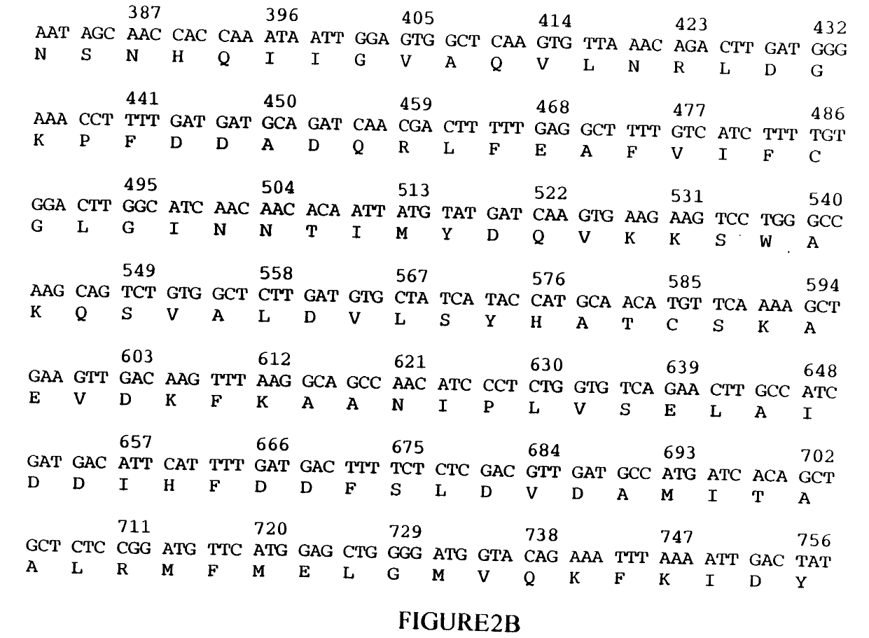

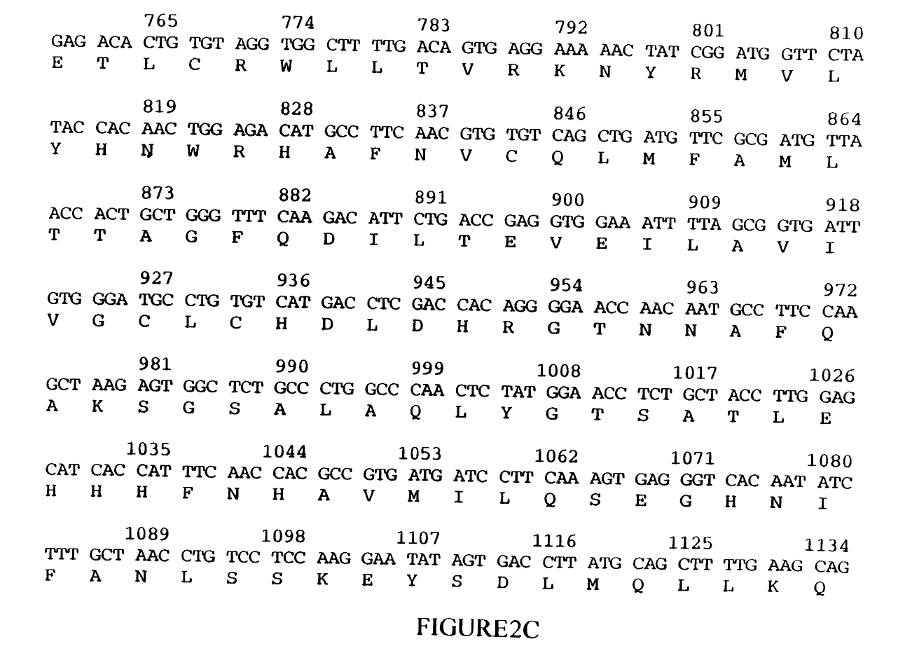

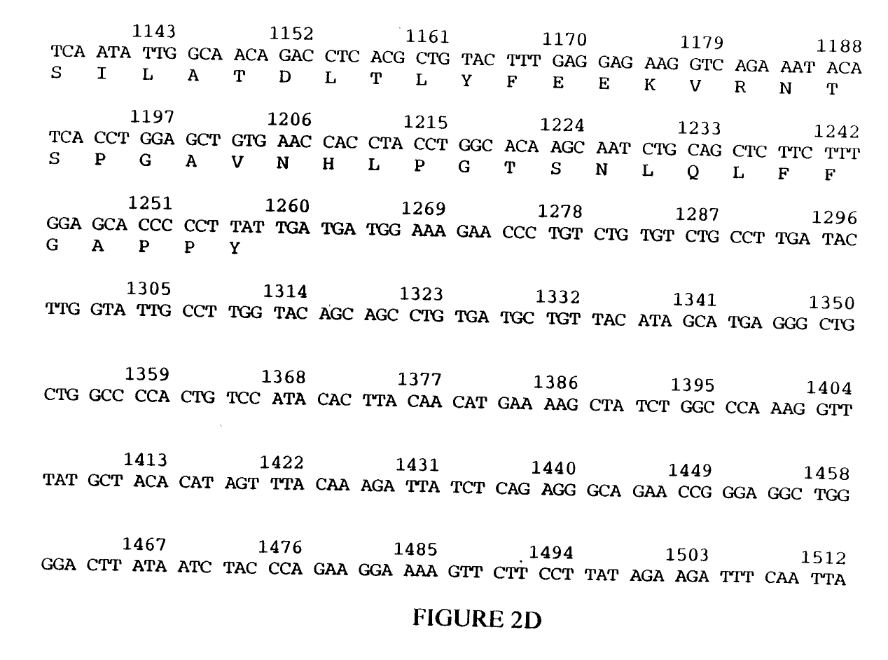

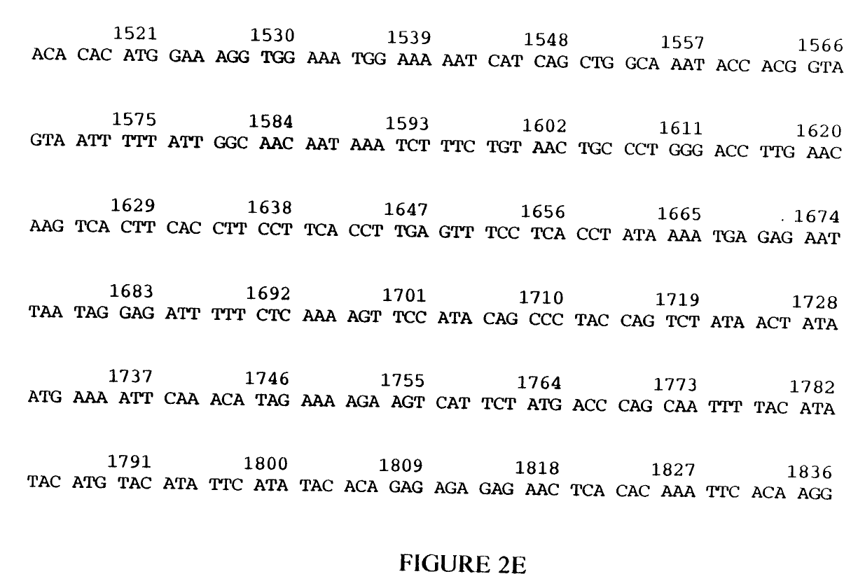

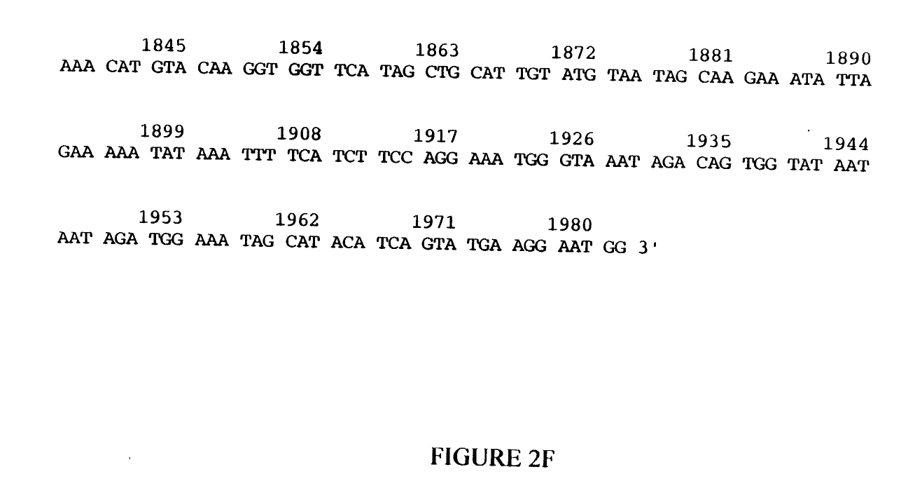

In another embodiment, the invention encompasses a protein comprising the amino acid sequence of SEQ ID NO:3. As shown in FIGS. 2A-2E, HSPDE10A2 is 367 amino acids in length and also contains the putative cGMP binding motif at N₈₈RLDGKPFDDAD of SEQ ID NO:3 and a PDE signature motif at H₂₆₀DLDHRGTNN of SEQ ID NO:3. As shown in FIGS. 3A-3E, HSPDE10A2 is identical to HSPDE10A1 between residues M₁ and E₃₃₈, but differs in the C-terminal portion of the molecule from E₃₃₉ to Y₃₆₇. HSPDE10A2 also shares 40% identity with HSPDE5A1.

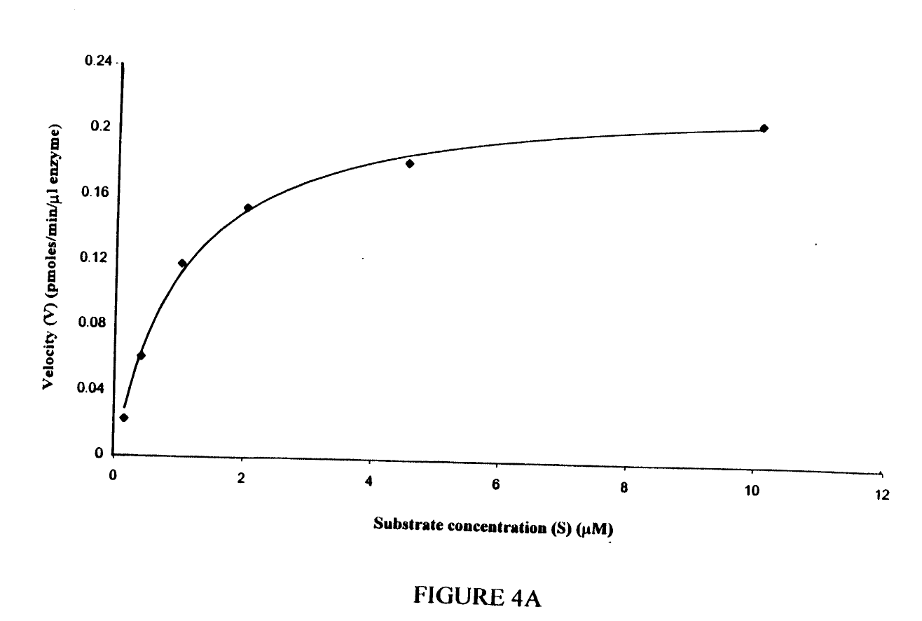

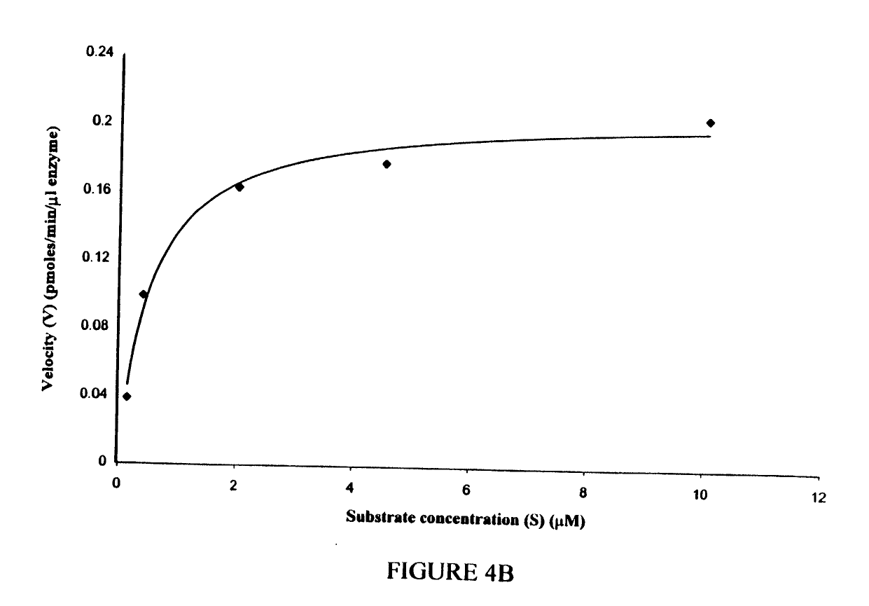

A cDNA construct encoding the full length amino acid sequence of HSPDE10A1 was cloned into the baculovirus transfer vector pFASTBAC, expressed in Sf9 cells, and the enzyme partially purified from these cells for enzyme assays. FIGS. 4A and 4B show the kinetics of HSPDE10A1 enzyme activity with cAMP (FIG. 4A) and cGMP (FIG. 4B) as substrates. Both substrates are hydrolyzed at a similar rate (Vₘₐₓ=0.23 and 0.21 μmole/min/ml enzyme for cAMP and cGMP, respectively), and with a similar affinity for HSPDE10A1 (Kₘ=1.04 and 0.52 μM for cAMP and cGMP, respectively). The data confirms that HSPDE10A1 is a PDE capable of hydrolyzing both cAMP and cGMP at physiologically relevant concentrations. The effects of various known PDE inhibitors on the activity of HSPDE10A1 using cGMP as a substrate are shown in Table 1. HSPDE10A1 was relatively insensitive to both milrinone and rolipram, which are selective for PDE3 and PDE4 respectively, with IC₅₀ values of >200 μM and 160 μM, respectively. The non-selective PDE inhibitor IBMX (3-isobutyl-1-methylxanthine) inhibited HSPDE10A1 with an IC₅₀ of 40 μM, which is within the range observed for other PDEs, except the IBMX-insensitive PDE8. The so-called cGMP PDE-specific inhibitor zaprinast, which is selective for PDE5 and PDE6, was moderately effective against HSPDE10A1 with an IC₅₀ of 8 μM (10-40 fold higher than PDEs 5 and 6).

The degree of similarity exhibited between the HSPDE10A1 and representatives of the other families of PDEs in the catalytic domain (25% to 50%) is consistent with that demonstrated between different PDE families (˜30%). HSPDE10A1 is further distinguished from other known families by its dual specificity for both cAMP and cGMP and by its pattern of inhibition by known PDE inhibitors. HSPDE10A1 therefore appears to be a member of a new family of cyclic nucleotide phosphodiesterases designated PDE10.

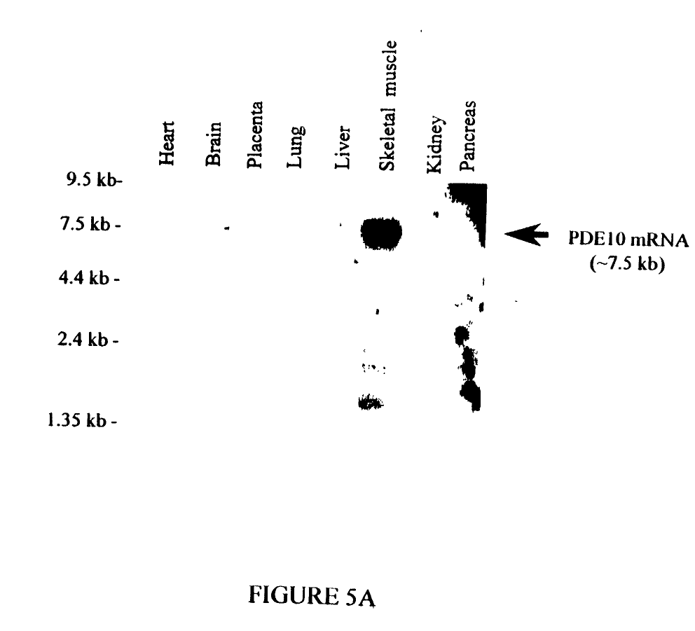

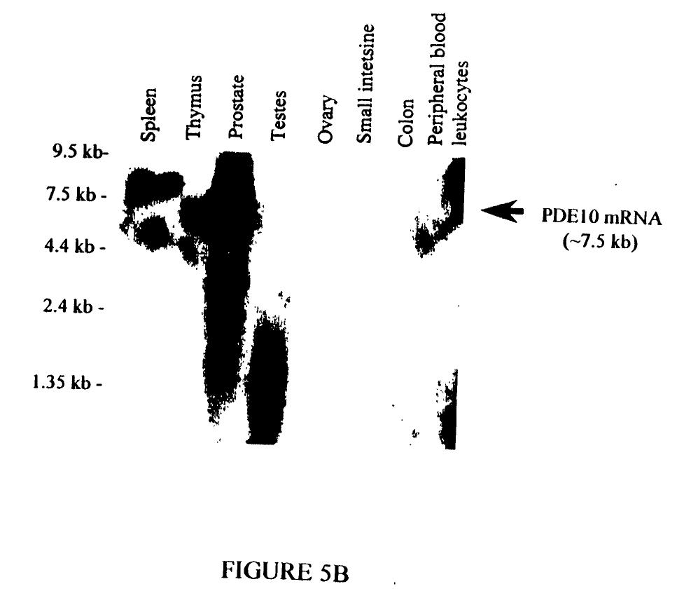

Membrane-based northern analysis (FIGS. 5A-5B) shows the expression of HSPDE10A as a major transcript of ˜7.5 kb in skeletal muscle and prostate tissue. An additional ˜3.0 kb mRNA was detected in prostate alone; a less prominent transcript of ˜1.5 kb occurs in skeletal muscle and testes. These data suggest the existence of at least three HSPDE10A splice variants. Electronic northern analysis using the LIFESEQ database (Incyte Genomics) further shows the expression of HSPDE10A in cancerous prostate tissue.

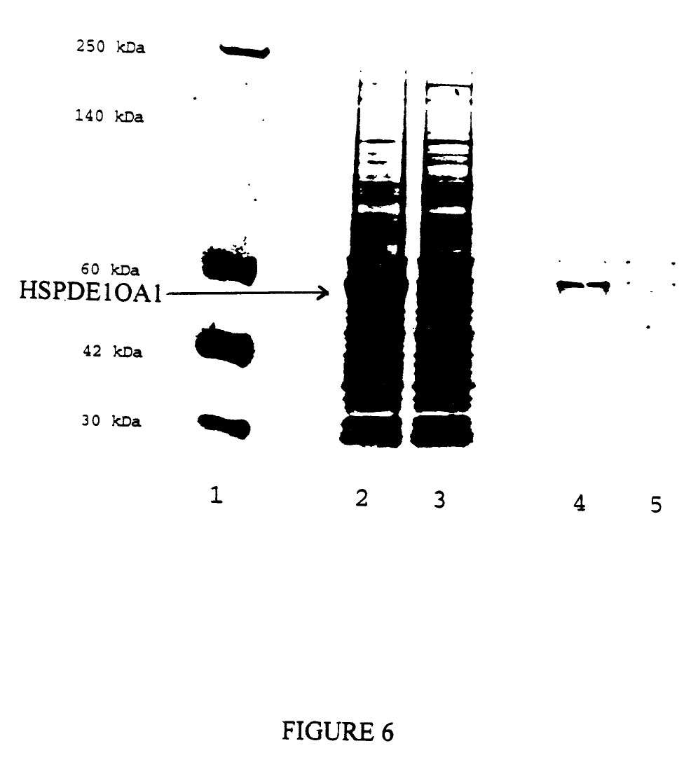

FIG. 6 shows the expression of HSPDE10A1 in cell lysates of Sf9 cells transformed with a baculovirus vector containing an untagged cDNA construct. An ˜56 kDa protein was detected by Coomassie blue staining (native HSPDE10A1; FIG. 6) and by western immunoblotting of a FLAG-tagged HSPDE10A1 using an anti-FLAG antibody (data not shown).

The invention also encompasses polynucleotides which encode HSPDE10A. In a particular embodiment, the invention encompasses a polynucleotide comprising the nucleic acid sequence of SEQ ID NO:2 which encodes HSPDE10A1. In another embodiment, the invention encompasses a polynucleotide comprising the nucleic acid sequence of SEQ ID NO:4 which encodes HSPDE10A2.

It will be appreciated by those skilled in the art that as a result of the degeneracy of the genetic code, a multitude of polynucleotides encoding HSPDE10A, some bearing minimal similarity to the polynucleotides of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring HSPDE10A, and all such variations are to be considered as being specifically disclosed.

Although nucleotide sequences which encode HSPDE10A and its variants are preferably capable of hybridizing to the nucleotide sequence of the naturally occurring HSPDE10A under appropriately selected conditions of stringency, it may be advantageous to produce nucleotide sequences encoding HSPDE10A or its derivatives possessing a different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for altering the nucleotide sequence encoding HSPDE10A and its derivatives without altering the encoded amino acid sequences include the production of RNA transcripts having more desirable properties, such as a greater half-life, than transcripts produced from the naturally occurring sequence.

The invention also encompasses production of DNA sequences which encode HSPDE10A and HSPDE10A derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding HSPDE10A.

Also encompassed by the invention are polynucleotides that are capable of hybridizing to the claimed polynucleotides, and, in particular, to those shown in SEQ ID NO:2, SEQ ID NO:4 or fragments thereof, under various conditions of stringency (Wahl and Berger (1987) Methods Enzymol 152:399-407; Kimmel (1987) Methods Enzymol 152:507-511). For example, stringent salt concentration will ordinarily be less than about 750 mM NaCl and 75 mM trisodium citrate (SSC), preferably less than about 500 mM NaCl and 50 mM SSC, and most preferably less than about 250 mM NaCl and 25 mM SSC. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM SSC, and 1% SDS. In a more preferred embodiment, hybridization will occur at 37° C. in 500 mM NaCl, 50 mM SSC, 1% SDS, 35% formamide, and 100 μg/ml denatured salmon sperm DNA (ssDNA). In a most preferred embodiment, hybridization will occur at 42° C. in 250 mM NaCl, 25 mM SSC, 1% SDS, 50% formamide, and 200 μg/ml ssDNA. Useful variations on these conditions will be readily apparent to those skilled in the art.

The washing steps which follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM SSC, and most preferably less than about 15 mM NaCl and 1.5 mM SSC. Stringent temperature conditions for the wash steps will ordinarily include temperature of at least about 25° C., more preferably of at least about 42° C., and most preferably of at least about 68° C. In a preferred embodiment, wash steps will occur at 25° C. in 30 mM NaCl, 3 mM SSC, and 0.1% SDS. In a more preferred embodiment, wash steps will occur at 42° C. in 15 mM NaCl, 1.5 mM SSC, and 0.1% SDS. In a most preferred embodiment, wash steps will occur at 68° C. in 15 mM NaCl, 1.5 mM SSC, and 0.1% SDS. Additional variations on these conditions will be readily apparent to those skilled in the art.

Methods for DNA sequencing are well known in the art and may be used to practice any of the embodiments of the invention. The methods may employ such enzymes as the Klenow fragment of DNA polymerase I, SEQUENASE, Taq polymerase, thermostable T7 polymerase (Arnersham Pharmacia Biotech (APB), Piscataway N.J.), or combinations of polymerases and proofreading exonucleases such as those found in the ELONGASE amplification system (Invitrogen, Carlsbad Calif.). Preferably, sequence preparation is automated with machines such as the HYDRA microdispenser (Robbins Scientific, Sunnyvale Calif.), MICROLAB 2200 system (Hamilton, Reno Nev.), DNA ENGINE thermal cycler (MJ Research, Watertown Mass.) and the CATALYST 800 thermal cycler (Applied Biosystems (ABI), Foster City Calif.). Sequencing is then carried out using either 373 or 377 DNA sequencing systems (ABI) or the MEGABACE 1000 DNA sequencing system (APB). The resulting sequences are analyzed using a variety of algorithms which are well known in the art and reviewed in Ausubel (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

The nucleic acid sequences encoding HSPDE10A may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar (1993) PCR Methods Applic 2:318-322). Another method, inverse PCR, uses primers that extend in divergent directions to amplify unknown sequence from a circularized template. The template is derived from restriction fragments comprising a known genomic locus and surrounding sequences (Triglia et al. (1988) Nucleic Acids Res 16:8186). A third method, capture PCR, involves PCR amplification of DNA fragments adjacent to known sequences in human and yeast artificial chromosome DNA (Lagerstrom et al. (1991) PCR Methods Applic 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker et al. (1991) Nucleic Acids Res 19:3055-306). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO software (Molecular Insights, Cascade Colo.) or another appropriate program, to be about 22 to 30 nucleotides in length, to have a GC content of about 50% or more, and to anneal to the template at temperatures of about 68° C. to 72° C.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using GENOTYPER or SEQUENCE NAVIGATOR software (ABI), and the entire process from loading of samples to computer analysis and electronic data display may be computer controlled. Capillary electrophoresis is preferable for sequencing small DNA fragments which may be present in limited amounts in a particular sample.

In another embodiment of the invention, polynucleotides or fragments thereof which encode HSPDE10A may be cloned in recombinant DNA molecules that direct expression of HSPDE10A, or portions or functional equivalents thereof, in appropriate host cells. Due to the inherent degeneracy of the genetic code, other DNA sequences which encode the same or a functionally equivalent amino acid sequence may be produced and used to express HSPDE10A.

The nucleotide sequences of the present invention can be engineered using methods generally known in the art in order to alter HSPDE10A-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In another embodiment, sequences encoding HSPDE10A may be synthesized, in whole or in part, using chemical methods well known in the art and described by Caruthers et al. (1980, Nucleic Acids Symp Ser (7) 215-223) and Horn et al. (1980, Nucleic Acids Symp Ser (7) 225-232). Alternatively, HSPDE10A itself or a portion thereof may be synthesized using chemical methods. For example, peptide synthesis can be performed using various solid-phase techniques (Roberge et al. (1995) Science 269:202-204). Automated synthesis may be achieved using the 431A Peptide synthesizer (ABI). Additionally, the amino acid sequence of HSPDE10A, or any part thereof, may be altered during direct synthesis and/or combined with sequences from other proteins, or any part thereof, to produce a variant protein.

The peptide may be purified by preparative high performance liquid chromatography. (Chiez and Regnier (1990) Methods Enzymol 182:392-421). The composition of the synthetic peptides may be confirmed by amino acid analysis or by sequencing. (See, e.g., Creighton (1984) Proteins, Structures and Molecular Properties, W H Freeman, New York N.Y.)

In order to express a biologically active HSPDE10A, the nucleotide sequences encoding HSPDE10A or derivatives thereof may be inserted into an appropriate expression vector, i.e., a vector which contains the necessary elements for transcriptional and translational control of the inserted coding sequence in a host. These elements include regulatory sequences, such as enhancers, constitutive and inducible promoters, and 5′ and 3′ untranslated regions in the vector and in polynucleotides encoding HSPDE10A. Such elements may vary in their strength and specificity. Specific initiation signals may also be used to achieve more efficient translation of sequences encoding HSPDE10A. Such signals include the ATG initiation codon and adjacent sequences, e.g. the Kozak sequence. In cases where sequences encoding HSPDE10A and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf et al. (1994) Results Probl Cell Differ 20:125-162).

Methods which are well known to those skilled in the art may be used to construct expression vectors containing sequences encoding HSPDE10A and appropriate transcriptional and translational control elements. These methods include in vitro recombinant DNA techniques, synthetic techniques, and in vivo genetic recombination as described in Sambrook et al. (1989; Molecular Cloning, A Laboratory Manual, Cold Spring Harbor Press, Plainview N.Y., ch. 4, 8, and 16-17) or in Ausubel et al. (1995; Current Protocols in Molecular Biology, John Wiley & Sons, New York N.Y., ch. 9, 13, and 16).

A variety of expression vector/host systems may be utilized to contain and express sequences encoding HSPDE10A. These include, but are not limited to, microorganisms such as bacteria transformed with recombinant bacteriophage, plasmid, or cosmid DNA expression vectors; yeast transformed with yeast expression vectors; insect cell systems infected with baculovirus expression vectors; plant cell systems transformed with viral expression vectors (cauliflower mosaic virus, CaMV, or tobacco mosaic virus, TMV) or with bacterial expression vectors (Ti or pBR322 plasmids); or animal cell systems. The invention is not limited by the host cell employed.

In bacterial systems, a number of cloning and expression vectors may be selected depending upon the use intended for polynucleotide sequences encoding HSPDE10A. For example, routine cloning, subcloning, and propagation of polynucleotides encoding HSPDE10A can be achieved using a multifunctional E. coli vector such as pBLUESCRIPT (Stratagene, La Jolla Calif.) or pSPORT1 plasmid (Invitrogen). Ligation of sequences encoding HSPDE10A into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke and Schuster (1989) J Biol Chem 264:5503-5509). When large quantities of protein are needed for the production of antibodies, vectors containing the strong, inducible T5 or T7 bacteriophage promoter which direct high level expression of may be used.

Yeast expression systems may be used for production of HSPDE10A. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation (Grant et al. (1987) Methods Enzymol 153:516-54; Scorer et al. (1994) Biotechnol 12:181-184).

Plant systems may also be used for expression of HSPDE10A. Transcription of sequences encoding HSPDE10A may be driven by viral promoters such as the 35S and 19S promoters of CaMV used alone or in combination with the omega leader sequence from TMV (Takamatsu (1987) EMBO J 6:307-311). Alternatively, plant promoters such as the small subunit of RUBISCO or heat shock promoters may be used (Coruzzi et al. (1984) EMBO J 3:1671-1680; Broglie et al. (1984) Science 224:838-843; and Winter et al. (1991) Results Probl Cell Differ 17:85-105). These constructs can be introduced into plant cells by direct DNA or pathogen-mediated transformation (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, sequences encoding HSPDE10A may be ligated into an adenovirus transcription/translation complex consisting of the late promoter and tripartite leader sequence. Insertion in a non-essential E1 or E3 region of the viral genome may be used to obtain infective virus which expresses HSPDE10A in host cells (Logan and Shenk (1984) Proc Natl Acad Sci 81:3655-3659). In addition, transcription enhancers, such as the RSV enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington et al. (1997) Nature Genet 15:345-355).

For long term production of recombinant proteins in mammalian systems, stable expression of HSPDE10A in cell lines is preferred. For example, sequences encoding HSPDE10A can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ or apr⁻ cells, respectively (Wigler et al. (1977) Cell 11:223-232; Lowy et al. (1980) Cell 22:817-823). Also, antimetabolite, antibiotic, or herbicide resistance can be used as the basis for selection. For example, dhfr confers resistance to methotrexate; neo confers resistance to the aminoglycosides, neomycin and G-418; and als or pat confer resistance to chlorsulfuron and phosphinotricin acetyltransferase, respectively (Wigler et al. (1980) Proc Natl Acad Sci 77:3567-3570; Colbere-Garapin et al. (1981) J Mol Biol 150:1-14). Additional selectable genes have been described, trpB and hisD, which alter cellular requirements for metabolites (Hartman and Mulligan (1988) Proc Natl Acad Sci 85:8047-8051). Visible markers, e.g., anthocyanins, GFP, β glucuronidase and its substrate β-glucuronide, or luciferase and its substrate luciferin may be used. These markers can be used not only to identify transformants, but also to quantify the amount of transient or stable expression attributable to a specific vector system (Rhodes (1995) Methods Mol Biol 55:121-131).

Although the presence/absence of marker gene expression suggests that the gene of interest is also present, the presence and expression of the gene may need to be confirmed. For example, if the sequence encoding HSPDE10A is inserted within a marker gene sequence, transformed cells containing sequences encoding HSPDE10A can be identified by the absence of marker gene function. Alternatively, a marker gene can be placed in tandem with a sequence encoding HSPDE10A under the control of a single promoter. Expression of the marker gene in response to induction or selection usually indicates expression of the tandem gene as well.

In general, host cells that contain the nucleic acid sequence encoding HSPDE10A and that express HSPDE10A may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip-based technologies for the detection and/or quantification of nucleic acid sequences.

A wide variety of labels and conjugation techniques are known by those skilled in the art and may be used in various nucleic acid and amino acid assays. Means for producing labeled hybridization or PCR probes for detecting sequences related to polynucleotides encoding HSPDE10A include oligolabeling, nick translation, end-labeling, or PCR amplification using a labeled nucleotide. Alternatively, the sequences encoding HSPDE10A, or any portions thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits and reporter molecules or labels.

Host cells transformed with nucleotide sequences encoding HSPDE10A may be cultured under conditions for the expression and recovery of the protein from cell culture. The protein produced by a transformed cell may be secreted or retained intracellularly depending on the sequence and/or the vector used. As will be understood by those of skill in the art, expression vectors containing polynucleotides which encode HSPDE10A may be designed to contain signal sequences which direct secretion of HSPDE10A through a prokaryotic or eukaryotic cell membrane.

In addition, a host cell strain may be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein may also be used to specify protein targeting, folding, and/or activity. Different host cells which have specific cellular machinery and characteristic mechanisms for post-translational activities (CHO, HeLa, MDCK, HEK293, WI38 and the like) are available from the ATCC (Manassas Va.) and may be chosen to ensure the correct modification and processing of the recombinant protein.

In another embodiment of the invention, natural, modified, or recombinant nucleic acid sequences encoding HSPDE10A may be ligated to a heterologous sequence resulting in translation of a fusion protein in any of the host systems. For example, a chimeric HSPDE10A protein containing a heterologous moiety that can be recognized by a commercially available antibody may facilitate the screening of peptide libraries for inhibitors of HSPDE10A activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the HSPDE10A encoding sequence and the heterologous protein sequence, so that HSPDE10A may be cleaved away from the heterologous moiety following purification. Methods for fusion protein expression and purification are discussed in Ausubel (1995, supra, ch 10). A variety of commercially available kits may also be used to facilitate expression and purification of fusion proteins.

In a further embodiment of the invention, synthesis of radiolabeled HSPDE10A may be achieved in vitro using the TNT rabbit reticulocyte lysate or wheat germ extract systems (Promega). These systems couple transcription and translation of protein-coding sequences operably associated with the T7, T3, or SP6 promoters. Translation takes place in the presence of a radiolabeled amino acid precursor, preferably ³⁵S-methionine.

Portions of HSPDE10A may be produced not only by recombinant production, but also by direct peptide synthesis using solid-phase techniques (Creighton, supra, pp. 55-60). Protein synthesis may be performed by manual techniques or by automation. Automated synthesis may be achieved, for example, using the 431A peptide synthesizer (ABI). Various portions of HSPDE10A may be synthesized separately and then combined to produce the full length molecule.

Antibodies

Antibodies, or immunoglobulins (Ig), are components of immune response expressed on the surface of or secreted into the circulation by B cells. The prototypical antibody is a tetramer composed of two identical heavy polypeptide chains (H-chains) and two identical light polypeptide chains (L-chains) interlinked by disulfide bonds which binds and neutralizes foreign antigens. Based on their H-chain, antibodies are classified as IgA, IgD, IgE, IgG or IgM. The most common class, lgG, is tetrameric while other classes are variants or multimers of the basic structure.

Antibodies that specifically bind HSPDE10A are described in terms of their two main functional domains. Antigen recognition is mediated by the Fab (antigen binding fragment) region of the antibody, while effector functions are mediated by the Fc (crystallizable fragment) region. The binding of antibody to antigen triggers destruction of the antigen by phagocytic white blood cells such as macrophages and neutrophils. These cells express surface Fc receptors that specifically bind to the Fc region of the antibody and allow the phagocytic cells to destroy antibody-bound antigen. Fc receptors are single-pass transmembrane glycoproteins containing about 350 amino acids whose extracellular portion typically contains two or three Ig domains (Sears et al. (1990) J Immunol 144:371-378).

Preparation and Screening of Antibodies

Various hosts including mice, rats, rabbits, goats, llamas, camels, and human cell lines may be immunized by injection with an antigenic determinant. Oligopeptides having from about 5 to about 15 amino acids and selected from the antigenic regions of SEQ ID NOs:1 and 3 may be used to induce antibody production. These oligopeptides which are identical to epitopes of the natural protein are fused with those of another protein, such as keyhole limpet hemacyanin (KLH; Sigma-Aldrich, St. Louis Mo.), and antibodies produced to the chimeric molecule. Adjuvants such as Freund's, mineral gels, and surface active substances such as lysolecithin, pluronic polyols, polyanions, peptides, oil emulsions, and dinitrophenol may be used to increase immunological response. In humans, BCG (bacilli Calmette-Guerin) and Corynebacterium parvum are preferable. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopepetides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al (1975) Nature 256:495-497; Kozbor et al (1985) J Immunol Methods 81:31-42; Cote et al (1983) Proc Natl Acad Sci 80:2026-2030; and Cole et al (1984) Mol Cell Biol 62:109-120).

Chimeric antibodies may be produced by techniques such as splicing of mouse antibody genes to human antibody genes to obtain a molecule with appropriate antigen specificity and biological activity (Morrison et al. (1984) Proc Natl Acad Sci 81:6851-6855; Neuberger et al. (1984) Nature 312:604-608; and Takeda et al. (1985) Nature 314:452-454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determinant may also be produced. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al (1989) Science 246:1275-1281).

Antibodies may also be produced by inducing production in the lymphocyte population or by screening immunoglobulin libraries or panels of highly specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are preferred for use in immunoassays in which the protein-antibody complex must withstand rigorous manipulations. Low-affinity antibody preparations with Kₐ ranging from about 10⁶ to 10⁷ L/mole are preferred for use in immunopurification and similar procedures which ultimately require dissociation of the protein, preferably in active form, from the antibody (Catty (1988) Antibodies, Volume I: A Practical Approach, IRL Press, Washington DC; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/ml, is generally employed in procedures requiring precipitation of protein-antibody complexes. Procedures for making antibodies, evaluating antibody specificity, titer, and avidity, and guidelines for antibody quality and usage in various applications, are widely available (Catty (supra); Ausubel (supra) pp. 11.1-11.31).

Diagnostics

In another embodiment, antibodies that specifically bind HSPDE10A and polynucleotides or fragments thereof that encode HSPDE10A or a portion thereof may be used for the diagnosis of disorders such as adenofibromatous hyperplasia of the prostate which is characterized by differential expression of HSPDE10A or in assays to monitor patients being treated with HSPDE10A or agonists, antagonists, or inhibitors of HSPDE10A.

Diagnostic assays for HSPDE10A include methods which utilize the antibody or polynucleotide and a reporter molecule to detect HSPDE10A in human body fluids or in extracts of cells or tissues. Such assays can be used for diagnosing cancers including adenocarcinoma, leukemia, lymphoma, melanoma, myeloma, sarcoma, and teratocarcinoma of the blood, bone, bone marrow, brain, colon, esophagus, ganglia, heart, kidney, liver, lung, muscle, nerve, ovaries, pancreas, prostate, small intestine, spleen, stomach, testis, and uterus, and immune disorders such as adult respiratory distress syndrome, asthma, atherosclerosis, cholecystitis, Crohn's disease, emphysema, multiple sclerosis, myasthenia gravis, osteoarthritis, osteoporosis, rheumatoid arthritis, scleroderma, systemic lupus erythematosus, and ulcerative colitis.

Normal or standard values for HSPDE10A expression are established by combining body fluids or cell extracts taken from a normal mammalian or human subject with antibodies or polynucleotides under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Immunological Assays

Immunological methods for detecting and measuring complex formation as a measure of protein expression using antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. A two-site, monoclonal-based immunoassay utilizing antibodies reactive to two non-interfering epitopes is preferred, but a competitive binding assay may be employed (Pound (1998) Immunochemical Protocols, Humana Press, Totowa N.J.).

Recently, antibody arrays have allowed the development of techniques for high-throughput screening of recombinant antibodies. Such methods use robots to pick and grid bacteria containing antibody genes, and a filter-based ELISA to screen and identify clones that express antibody fragments. Because liquid handling is eliminated and the clones are arrayed from master stocks, the same antibodies can be spotted multiple times and screened against multiple antigens simultaneously. Antibody arrays are highly useful in the identification of differentially expressed proteins. (See de Wildt et al. (2000) Nature Biotechnol 18:989-94.)

Nucleic Acid Assays

Polynucleotides which may be used include oligonucleotide sequences, complementary RNA and DNA molecules, and peptide nucleic acids. The polynucleotides may be used to detect and quantitate gene expression in biopsied tissues in which expression of HSPDE10A may be correlated with cancer or immune disorder. The diagnostic assay may be used to determine differential expression of HSPDE10A in a disease process or to monitor regulation of HSPDE10A levels during therapeutic intervention. The polynucleotides encoding HSPDE10A may be used in Southern or northern analysis, dot blot, or other membrane-based technologies; in PCR technologies; in dipstick, pin, and multiwell formats; and in microarrays utilizing fluids or tissues from patients to detect altered HSPDE10A expression. Such qualitative or quantitative methods are well known in the art.

In one aspect, hybridization with PCR probes which are capable of detecting mRNAs encoding HSPDE10A may be used. The specificity of the probe, whether it is made from a highly specific region, such as the 5′ regulatory region, or from a less specific region, such as a conserved motif, and the stringency of the hybridization or amplification (maximal, high, intermediate, or low), will determine whether the probe identifies only naturally occurring sequences encoding HSPDE10A, allelic variants, or related sequences.

Probes which may be used for the detection of related sequences should preferably have at least 50% sequence identity to any of the HSPDE10A encoding sequences. The hybridization probes of the subject invention may be DNA or RNA, may be derived from SEQ ID NO:2 or 4 or from introns of HSPDE10A.

Means for producing specific hybridization probes for DNAs encoding HSPDE10A include the cloning of polynucleotides encoding HSPDE10A or HSPDE10A derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter molecules.

With respect to cancer, the presence of an abnormal amount of transcript (either under- or over-expressed) in biopsied tissue from an individual may indicate a predisposition for the development of the disease, or may provide a means for detecting the disease prior to the appearance of actual clinical symptoms. A more definitive diagnosis of this type may allow health professionals to employ aggressive treatment earlier thereby preventing the development or further progression of the cancer.

Additional diagnostic uses for oligonucleotides designed from the sequences encoding HSPDE10A may involve the use of PCR. These oligomers may be chemically synthesized, generated enzymatically, or produced in vitro. Oligomers will preferably contain a fragment of a polynucleotide encoding HSPDE10A, or a fragment of a polynucleotide complementary to the polynucleotide encoding HSPDE10A, and will be employed under optimized conditions for identification-of a specific gene or disorder. Oligomers may also be employed under less stringent conditions for detection or quantitation of closely related DNA or RNA sequences.

In further embodiments, polynucleotides their fragments or oligonucleotides may be used on a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Microarrays may be prepared, used, and analyzed using methods known in the art. (See, e.g., Brennan et al. (1995) U.S. Pat. No. 5,474,796; Schena et al. (1996) Proc Natl Acad Sci 93:10614-10619; Baldeschweiler et al. (1995) PCT application WO95/251116; Shalon et al. (1995) PCT application WO95/35505; Heller et al. (1997) Proc Natl Acad Sci 94:2150-2155; and Heller et al. (1997) U.S. Pat. No. 5,605,662.)

Mapping of new polynucleotides to chromosomal arms may provide valuable information to investigators searching for disease genes using positional cloning or other gene discovery techniques. Once the disease or syndrome has been crudely localized by genetic linkage to a particular genomic region, e.g., ataxia-telangiectasia to 11q22-23, any sequences mapping to that area may represent associated or regulatory genes for further investigation (Gatti et al. (1988) Nature 336:577-580). The nucleotide sequence of the subject invention may also be used to detect differences in the chromosomal location due to translocation, inversion, deletion and the like among normal, carrier, or affected individuals.

Therapeutics

Chemical and structural similarity in the context of sequences and motifs exists between regions of HSPDE10A and human cyclic nucleotide phosphodiesterases. In addition, the expression of HSPDE10A is closely associated with normal skeletal muscle and prostate tissues. Therefore, HSPDE10A appears to be downregulated in prostate cancer, specifically in adenofibromatous hyperplasia. In the treatment of this and other disorders associated with decreased HSPDE10A expression or activity, it is desirable to increase the expression or activity of HSPDE10A by delivery of the protein, a vector expressing the protein or an agonist of HSPDE10A. In those disorders in which the increased expression of the protein is implicated in the cancerous or immune disease process, it is desirable to decrease expression or activity of HSPDE10A by delivery of an antibody, antagonist, or inhibitor.

Therefore, in one embodiment, HSPDE10A or a portion or derivative thereof may be administered to a subject to treat a disorder associated with decreased expression or activity of HSPDE10A. Examples of such disorders include, but are not limited to, a cancer, such as cancer such as adenocarcinoma, leukemia, lymphoma, melanoma, myeloma, sarcoma, and teratocarcinoma of the blood, bone, bone marrow, brain, colon, esophagus, ganglia, heart, kidney, liver, lung, muscle, nerve, ovaries, pancreas, prostate, small intestine, spleen, stomach, testis, and uterus and immune disorders such as adult respiratory distress syndrome, asthma, atherosclerosis, cholecystitis, Crohn's disease, emphysema, multiple sclerosis, myasthenia gravis, osteoarthritis, osteoporosis, rheumatoid arthritis, scleroderma, systemic lupus erythematosus, and ulcerative colitis.

In a second embodiment, a vector capable of expressing HSPDE10A or a portion or derivative thereof may be administered to a subject to treat a disorder associated with decreased expression or activity of HSPDE10A including, but not limited to, those described above.

In a third embodiment, a composition comprising a purified HSPDE10A in conjunction with a carrier may be administered to a subject to treat a disorder associated with decreased expression or activity of HSPDE10A including, but not limited to, those provided above.

In a fourth embodiment, a composition comprising an agonist that specifically binds HSPDE10A and which increases the expression, activity, or lifespan of HSPDE10A may be administered to a subject to treat a disorder associated with decreased expression or activity of HSPDE10A including, but not limited to, those listed above.

In a fifth embodiment, an antagonist of HSPDE10A may be administered to a subject to treat or prevent a disorder associated with increased expression or activity of HSPDE10A. Such disorders may include, but are not limited to, those discussed above. In one aspect, an antibody that specifically binds HSPDE10A may be used directly as an antagonist or indirectly as a targeting or delivery mechanism for bringing a pharmaceutical agent to cells or tissue which express HSPDE10A.

In a sixth embodiment, a vector expressing the complement of the polynucleotide encoding HSPDE10A may be administered to a subject to treat a disorder associated with increased expression or activity of HSPDE10A including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment of the various disorders described above. Using this approach, one may be able to achieve therapeutic efficacy with lower dosages of each agent, thus reducing the potential for adverse side effects.

An antagonist of HSPDE10A may be produced using methods which are generally known in the art. In particular, purified HSPDE10A may be used to produce antibodies or to screen libraries of pharmaceutical agents to identify those that specifically bind HSPDE10A. Therapeutic antibodies that specifically bind HSPDE10A may also be generated using methods that are well known in the art. Such antibodies may include, but are not limited to, polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein. Neutralizing antibodies are preferred for therapeutic use.

In another embodiment of the invention, the polynucleotides encoding HSPDE10A, or any fragment or complement thereof, may be used for therapeutic purposes. in one aspect, the complement of the polynucleotide encoding HSPDE10A may be used in situations in which it would be desirable to block the transcription of the mRNA. In particular, cells may be transformed with sequences complementary to polynucleotides encoding HSPDE10A. Thus, complementary molecules or fragments may be used to modulate HSPDE10A activity, or to achieve regulation of gene function. Such technology is now well known in the art, and sense or antisense oligonucleotides or larger fragments can be designed from various locations along the coding or control regions of sequences encoding HSPDE10A.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express nucleic acid sequences complementary to the polynucleotides encoding HSPDE10A. (See, e.g., Sambrook, supra; Ausubel, 1995, supra.)

Genes encoding HSPDE10A can be turned off by transforming a cell or tissue with expression vectors which express high levels of a polynucleotide, or fragment thereof, encoding HSPDE10A. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or peptide nucleic acids) to the control, 5′, or regulatory regions of the gene encoding HSPDE10A. Oligonucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described by Gee et al. (1994; In: Huber and Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding HSPDE10A.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding HSPDE10A. Such DNA sequences may be incorporated into a wide variety of vectors with RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of peptide nucleic acids and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods described by Goldman et al. (1997) Nature Biotechnol 15:462-466).

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical or sterile composition, in conjunction with a pharmaceutically acceptable carrier, for any of the therapeutic effects discussed above. Such compositions may consist of HSPDE10A, antibodies to HSPDE10A, and mimetics, agonists, antagonists, or inhibitors of HSPDE10A. The compositions may be administered alone or, which may be administered in any. The compositions may be administered to a patient alone, with a stabilizing compound, with a sterile, biocompatible carrier such as saline, buffered saline, dextrose, or water and in combination with other agents, drugs, or hormones.

Pharmaceutical Compositions

Pharmaceutical compositions may be formulated and administered, to a subject in need of such treatment, to attain a therapeutic effect. Such compositions contain the instant protein, agonists, antibodies specifically binding the protein, antagonists, inhibitors, or mimetics of the protein. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropylmethyl-cellulose, methyl cellulose, or sodium carboxymethylcellulose; gums including arabic and tragacanth; lubricants such as alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature and generally available to practitioners. Further details on techniques for formulation and administration may be found in the latest edition of Remington's Pharmaceutical Sciences (Mack Publishing, Easton Pa.).

In additional embodiments, HSPDE10A may be used in any molecular biology techniques that have yet to be developed, provided the new techniques rely on properties of nucleotide sequences that are currently known, including, but not limited to, such properties as the triplet genetic code and specific base pair interactions.

The examples below are provided to illustrate the subject invention and are not included for the purpose of limiting the invention.

### EXAMPLES

I cDNA Library Construction

The PROSNOT06 cDNA library was constructed from microscopically normal prostate tissue obtained from a 57-year-old Caucasian male. Pathology for the associated tumor indicated adenocarcinoma (Gleason grade 3+3) in both the left and right periphery of the prostrate. Perineural invasion was present as was involvement of periprostatic tissue. Patient history included a benign neoplasm of the large bowel, appendectomy, and tonsillectomy with adenoidectomy. Family history included a malignant neoplasm of the prostate and type I diabetes.

The frozen tissue was homogenized and lysed using a POLYTRON homogenizer (PT-3000; Brinkmann Instruments, Westbury N.J.) in guanidinium isothiocyanate solution. The lysate was extracted once with acid phenol per standard RNA isolation protocol (Stratagene). The RNA was extracted a second time with acid phenol, pH 4.7, precipitated using 0.3 M sodium acetate and 2.5 volumes of ethanol, resuspended in DEPC-treated water, and treated with DNAse at 37° C. for 25 minutes. mRNA was isolated with the OLIGOTEX kit (Qiagen, Chatsworth Calif.) and used to construct the cDNA libraries. cDNAs were fractionated on a SEPHAROSE CL4B column (APB), and those cDNAs exceeding 400 bp were ligated into pSPORT1 plasmid (Invitrogen). The plasmid was transformed into DH5α competent cells (Invitrogen).

II Isolation of cDNA Clones

Plasmid DNA was released from the cells and purified using the REAL PREP 96 plasmid kit (Qiagen). The recommended protocol was employed except for the following changes: 1) the bacteria were cultured in 1 ml of sterile TERRIFIC BROTH (BD Biosciences, San Jose Calif.) with carbenicillin at 25 mg/l and glycerol at 0.4%; 2) the cultures were incubated for 19 hours, and the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

III Sequencing and Analysis

The cDNAs were prepared for sequencing using the CATALYST 800 thermal cycler (ABI), or the HYDRA microdispenser (Robbins Scientific) or the MICROLAB 2200 (Hamilton) systems in combination with the DNA ENGINE thermal cyclers (MJ Research). The cDNAs were sequenced using the PRISM 373 or 377 sequencing systems and standard protocols, base calling software, and kits (ABI) or using the MEGABACE 1000 DNA sequencing system with standard solutions and dyes (APB). Reading frames were determined using standard methods as reviewed in Ausubel (1997, supra, unit 7.7). Some of the cDNA sequences were extended using the techniques disclosed in Example V.

The polynucleotides derived from cDNA, extension, and shotgun sequencing were assembled and analyzed using a combination of software programs which utilize algorithms well known to those skilled in the art. Table 2 summarizes the software programs, descriptions, references, and threshold parameters used. The first column of Table 2 shows the tools, programs, and algorithms used, the second column provides a brief description thereof, the third column presents the references which are incorporated by reference herein, and the fourth column presents, where applicable, the scores, probability values, and other parameters used to evaluate the strength of a match between two sequences (the higher the probability the greater the homology). Sequences were analyzed using MACDNASIS PRO software (Hitachi Software Engineering) and LASERGENE software (DNASTAR).

The polynucleotides were validated by removing vector, linker, and polyA sequences and by masking ambiguous bases, using algorithms and programs based on BLAST, dynamic programming, and dinucleotide nearest neighbor analysis. The sequences were then queried against a selection of public databases such as GenBank primate, rodent, mammalian, vertebrate, and eukaryote databases. and BLOCKS to acquire annotation, using programs based on BLAST, FASTA, and BLIMPS. The sequences were assembled into full length polynucleotides using programs based on Phred, Phrap, and Consed, and were screened for open reading frames using programs based on GeneMark, BLAST, and FASTA. The full length polynucleotides were translated to derive the corresponding full length amino acid sequences, and these full length sequences were subsequently analyzed by querying against databases such as the GenBank databases (described above), SwissProt, BLOCKS, PRINTS, PFAM, and Prosite.

The programs described above for the assembly and analysis of full length polynucleotide and amino acid sequences were used to identify fragments from SEQ ID NO:2 or SEQ ID NO:4. Fragments from about 20 to about 4000 nucleotides which are useful in hybridization and amplification technologies were described in The Invention section above.

IV Cloning of Full Length HSPDE10A

The complete cDNA insert from Incyte clone 826776 was isolated as a Sal1/Not1 restriction fragment, labeled with [α-³²P]dCTP, and used as a hybridization probe to screen ˜1×10⁶ plaque forming units from a human skeletal muscle 5′-STRETCH PLUS λgt10 cDNA library (Clontech). Each cDNA insert was recovered as an EcoRI restriction fragment(s) and subcloned into pBLUESCRIPT KS+ (Stratagene). One λ clone (clone 1a.1) contained a 3.9 kb cDNA insert. Identification of a single, large open reading frame (FIGS. 1A-1E) allowed sequencing of both strands to produce the consensus nucleotide sequence, SEQ ID NO:2. HSPDE10A2, a C-terminal splice variant of HSPDE10A2 was also isolated by hybridization screening of the human skeletal muscle cDNA library (Clontech). When the clone was isolated and fully sequenced, it revealed an insert with a 5′ coding region similar to HSPDE10A1 and a 3′ end similar to that of the original Incyte clone 826776 (FIGS. 2A-2F).

V Northern Analysis

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel, 1995, supra, ch. 4 and 16.)

Membrane-based northern analysis was performed on RNA samples from a variety of human tissues using Multiple Tissue Northern (MTN) blots (Clontech). For detecting human HSPDE10A, the ˜1 kb cDNA insert of Incyte clone 826776 (Sal1/Not1 restriction fragment) was used. This insert comprises 108 bp 5′ of the catalytic domain and 429 bp of the catalytic domain that is common to both HSPDE10A1 and HSPDE10A2. To examine HSPDE10A1 specifically, the ˜1.7 kb EcoRI restriction fragment of λ clone 1a.1 which comprises 447 bp of the 3′ portion of the catalytic domain and ˜1.2 kb of the 3′untranslated region was used.

Each probe was labeled with [α-³²P]dCTP using a MEGAPRIME kit (APB) and reaction products were purified using CHROMASPIN-30 columns (Clontech). The MTN blots were pre-hybridized in EXPRESSHYB (Clontech) at 68° C. for 1 hour and hybridized (˜1×10⁶ cpm probe/ml) at 68° C. overnight. Blots were washed in 2×SSPE, 0.05% (w/v) SDS at 50° C. (4×15 mins) followed by 0.1×SSPE, 0.1% (w/v) SDS at 50° C. for 1 hour, and then exposed to film for 2-7 days. Blots were checked for equal loading of poly(A)⁺ RNA in each lane using a human β-actin cDNA probe (data not shown).

Northern analysis showed that HSPDE10A was expressed in skeletal muscle and prostate as a major transcript of ˜7.5 kb; a ˜3.0 kb mRNA was detected only in prostate; and a less prominent transcript of ˜1.5 kb occurred in testes and skeletal muscle (FIGS. 5A and 5B). These data suggest that at least three PDE10A splice variants exist. Electronic northern analysis confirms expression in prostate (FIG. 7).

Analogous computer techniques applying BLAST were used to search for identical or related molecules in nucleotide databases such as GenBank or LIFESEQ database (Incyte Genomics). This analysis is much faster than multiple membrane-based hybridizations. In addition, the sensitivity of the computer search is modified to determine whether any particular match is categorized as exact or similar. The basis of the search is the product score, which is defined as:

$$\frac{\% sequence identity \times \% maximum BLAST score}{100}$$

The product score takes into account both the degree of similarity between two sequences and the length of the sequence match. For example, with a product score of 40, the match will be exact within a 1% to 2% error, and, with a product score of 70, the match will be exact. Similar molecules are usually identified by selecting those which show product scores between 15 and 40, although lower scores identify related molecules.

The results of northern analyses were reported as a percentage distribution of libraries in which the transcript encoding HSPDE10A occurred. Analysis involved the categorization of cDNA libraries by organ/tissue and disease. The organ/tissue categories included cardiovascular, dermatologic, developmental, endocrine, gastrointestinal, hematopoietic/immune, musculoskeletal, nervous, reproductive, and urologic. The disease categories included cancer, inflammation/trauma, fetal, neurological, and pooled. For each category, the number of libraries expressing the sequence of interest was counted and divided by the total number of libraries across all categories. Percentage values of tissue-specific and disease expression are reported in the description of the invention.

VI Labeling and Use of Individual Hybridization Probes

Hybridization probes derived from SEQ ID NO:2 and SEQ ID NO:4 are employed to screen cDNAs, genomic DNAs, or mRNAs. Although the labeling of oligonucleotides, consisting of about 20 base pairs, is specifically described, the same procedure is used with larger nucleotide fragments. Oligonucleotides are designed using state-of-the-art software such as OLIGO software (Molecular Insights) and labeled by combining 50 pmol of each oligomer, 250 μCi of [³²P]-adenosine triphosphate (APB), and T4 polynucleotide kinase (NEN Life Science Products, Boston Mass.). The labeled oligonucleotides are purified using a SEPHADEX G-25 superfine size exclusion dextran bead column (APB). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba1, or Pvu II (NEN Life Science Products).

The DNA from each digest is fractionated on a 0.7% agarose gel and transferred to a NYTRAN PLUS membranes (Schleicher & Schuell, Durham N.H.). Hybridization is carried out for 16 hours at 40° C. To remove nonspecific signals, blots are sequentially washed at room temperature under increasingly stringent conditions up to 0.1× saline sodium citrate and 0.5% sodium dodecyl sulfate. After XOMAT-AR film (Eastman Kodak, Rochester N.Y.) is exposed to the blots to film for several hours, hybridization patterns are compared visually.

VII Microarrays

A chemical coupling procedure and an ink jet device is used to synthesize array elements on the surface of a substrate (Baldeschweiler, supra). An array analogous to a dot or slot blot is used to arrange and link elements to the surface of a substrate using thermal, UV, chemical, or mechanical bonding procedures. A typical array is produced by hand or using available methods and machinery and contains any appropriate number of elements. After hybridization, nonhybridized probes are removed, and a scanner used to determine the levels and patterns of fluorescence. The degree of complementarity and the relative abundance of each probe which hybridizes to an element on the microarray is assessed through analysis of the scanned images.

Full-length cDNAs, expressed sequence tags (ESTs), or fragments thereof comprise the elements of the microarray. Fragments for hybridization are selected using software well known in the art such as LASERGENE software (DNASTAR). Full-length cDNAs, ESTs, or fragments thereof corresponding to one of the nucleotide sequences of the present invention, or selected at random from a cDNA library relevant to the present invention, are arranged on an appropriate substrate, e.g., a glass slide. The cDNA is fixed to the slide using UV cross-linking followed by thermal and chemical treatments and dried (Schena et al. (1995) Science 270:467-470; Shalon et al. (1996) Genome Res 6:639-645). Fluorescent probes are prepared and used for hybridization to the elements on the substrate. The substrate is analyzed by procedures described above.

VIII Complementary Polynucleotides

Sequences complementary to the HSPDE10A-encoding sequences are used to detect, decrease, or inhibit expression of naturally occurring HSPDE10A. Although use of oligonucleotides comprising from about 15 to 30 base pairs is described, the same procedure is used with smaller or with larger fragments. Appropriate oligonucleotides are designed using OLIGO software (Molecular Insights) and the coding sequence of HSPDE10A. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the HSPDE10A-encoding transcript.

IX Subcloning and Expression of HSPDE10A

Two constructs encoding full length human HSPDE10A1 enzyme (plus and minus an N-terminal epitope tag) were generated for expression in insect cells using a baculovirus vector. Full length human HSPDE10A1 was isolated by PCR from λ clone 1a.1 using a sense primer, 5′-CCAAATCCCGGTCCGAG-ATGTCCCCAAAGTGCAGTGCTGATGC-3′ (SEQ ID NO:6), covering the initiation codon (underlined) and incorporating an RsrII restriction enzyme site, and an antisense primer, 5′-CGGGTACCTCGAGTTA-TTAGTTCCTGTCTTCCTTGGCTACC-3′ (SEQ ID NO:7), covering the termination codon (underlined) and incorporating a tandem stop codon and unique XhoI restriction site. PCR was performed using the Expand High Fidelity PCR system (Boehringer Mannheim, West Sussex UK) and the following cycle conditions: 94° C./1′45″, 1 cycle; 94° C./15″, 65° C./30″, 72° C./1′45″, 20 cycles, and 72° C./5′, 1 cycle. The PCR product was digested with RsrII/XhoI and the resulting restriction fragment ligated into the RsrII/XhoI sites of the baculovirus transfer vector, pFASTBAC (Invitrogen), with and without modification to include a 5′FLAG epitope tag (Kunz et al. (1992) J Biol Chem 267:9101-9106). The sequence of the insert for each construct was determined on both strands to confirm identity to the native HSPDE10A1 coding sequence, the encoded sequence being either native HSPDE10A1 or N-terminally FLAG-tagged HSPDE10A1.

Recombinant viral stocks were prepared using the Bac-to-Bac system (Invitrogen) according to the manufacturer's protocol, and Sf9 cells were cultured in Sf 900 II serum-free media (Invitrogen) at 27° C. For expression, 3×10⁷ cells in 30 ml were infected at a multiplicity of infection of 1. Cells were harvested 48 hours post-infection for assay. HSPDE10A for enzyme activity assays was prepared from transformed Sf9 cells harvested and disrupted by sonication. Cellular debris was removed by centrifugation at 12,000×g for 15 mins followed by filtration (0.2 μm filter), and the clarified supernatant dialyzed against 20 mM HEPES pH 7.4, 1 mM EDTA, 150 mM NaCl at 4° C. overnight.

HSPDE10A1 was partially purified from the dialyzed supernatant by ion exchange chromatography using a 1 ml Mono Q HR (5/5) column (APB). The column was eluted using a linear NaCl gradient up to 1M, and fractions containing high activity (>70% substrate turnover) were pooled and stored in aliquots at −70° C.

X PAGE and Western Analysis of HSPDE10A

Transformed Sf9 cells were harvested by centrifugation (3,000×g for 10 min), resuspended in homogenization buffer (20 mM HEPES pH 7.2, 1 mM EDTA, 20 mM sucrose, 150 mM NaCl and containing one protease inhibitor tablet (Boehringer Mannheim) per 50 ml) at 1×10⁷ cells/ml and disrupted by sonication. Cellular debris was removed by centrifugation at 12,000×g for 15 min, and the supernatant stored in aliquots at −70° C.

Human PDE10A1 infected and mock infected (control) cell lysates (˜6.4×10⁴ cell equivalents for Coomassie staining, and ˜640 cell equivalents for western analysis) were separated by denaturing PAGE using the NuPAGE mini-gel system (Novex, San Diego Calif.) and either stained with Coomassie or transferred to a polyvinylidene difluoride membrane (Novex) for immunoblotting. Western analysis was performed by enhanced chemiluminescence (APB), according to the manufacturer's protocol using an anti-FLAG antibody (Sigma-Aldrich, Dorset UK) and a horse radish peroxidase conjugated anti-mouse IgG (Bio-Rad, Herts UK) as a secondary antibody at 1:500 and 1:1,000 dilutions, respectively.

XI Demonstration of HSPDE10A Activity

PDE activity of HSPDE10A1 was measured using a scintillation proximity assay (SPA)-based method employing the modification reported by Hurwitz et al. (1984; J Biol Chem 259:8612-8618). 50 μl of 20 mM Tris-HCl pH 7.4 and 5 mM MgCl₂ containing the required concentration of cyclic nucleotide was added to 50 μl of diluted enzyme (or no enzyme for background control) in 20 mM Tris-HCl pH 7.4, 5 mM MgCl₂ and 2 mg/ml bovine serum albumin to initiate the reaction. Both cAMP and cGMP were used as substrates (0.15-10 μM final concentration) with a 3:1 ratio of unlabeled to [³H]-labeled cAMP or cGMP (APB). Reactions were performed in triplicate in MICROFLUOR plates (Dynex Technologies, Chantilly Va.) at 30° C. for a period of time that would give less than 25% substrate turnover to avoid non-linearity associated with product inhibition. The reaction was terminated by the addition of 50 μl of PDE SPA beads (yttrium silicate, 20 mg/ml in water; APB) along with a large excess (1 mM final concentration) of the respective unlabeled cyclic nucleotide (cGMP or cAMP). Plates were sealed and shaken for 10 minutes to allow the beads to bind the nucleotide product. The SPA beads were allowed to settle for 30 minutes, and the plates were read on a TOPCOUNT plate reader (Packard Instrument, Meriden Conn.).

To determine the Kₘ and Vₘₐₓ of the enzyme, the rate of hydrolysis of cAMP and cGMP was measured at a variety of substrate concentrations (0.15-10 μM) using a fixed amount of diluted enzyme over a time-course of 5-60 minutes. Data points in the linear part of the reaction were used to calculate Kₘ and Vₘₐₓ from a Michaelis-Menten plot using the ‘Fit Curve’ program of EXCEL software (Microsoft, Redmond Wash.).

Inhibition studies were performed using the assay described above except that the appropriate inhibitor, dissolved and diluted as required in dimethylsulphoxide, was added to the diluted enzyme to give the required final concentration (1-200 μM). Reactions were initiated by the addition of substrate. Cyclic GMP was used as substrate at a final concentration of 0.17 μM, a concentration equal to ⅓ Kₘ so that IC₅₀˜Ki. Sufficient enzyme was added to give ˜25% substrate turnover during a 30 minute incubation at 30° C.

XII Functional Assays

HSPDE10A function is assessed by expressing the sequences encoding HSPDE10A at physiologically elevated levels in mammalian cell culture systems. cDNA is subcloned into pCMV SPORT (Invitrogen) which contains the cytomegalovirus promoter. 5-10 μg of recombinant vector 1-2 μg of an additional plasmid containing CD64-GFP fusion protein (Clontech) are transiently transformed into a human cell line, preferably of endothelial or hematopoietic origin, using either liposome formulations or electroporation. Flow cytometry (FCM) is used to identify transformed cells expressing CD64-GFP.

Transformed cells are collected by contacting the cells with CD64-GFP expressed on their surface with magnetic beads coated with human IgG (DYNAL, Lake Success N.Y.). mRNA is purified from the cells using methods described above, and expression of mRNA encoding HSPDE10A is analyzed and quantified by either northern analysis or microarray techniques.

XIII Production of HSPDE10A Specific Antibodies

HSPDE10A purified using PAGE (Harrington (1990) Methods Enzymol 182:488-495), is used to immunize rabbits and to produce antibodies. Alternatively, the HSPDE10A amino acid sequence is analyzed using LASERGENE software (DNASTAR) to determine regions of high immunogenicity, and an oligopeptide is synthesized and used to raise antibodies.

Typically, oligopeptides 15 residues in length are synthesized using an 431A Peptide synthesizer (ABI) using FMOC-chemistry and coupled to KLH (Sigma-Aldrich) by reaction with N-maleimidobenzoyl-N-hydroxysuccinimide ester to increase immunogenicity (Ausubel, supra). Rabbits are immunized with the oligopeptide-KLH complex in complete Freund's adjuvant. Resulting antisera are tested for antipeptide activity by binding the peptide to plastic, blocking with 1% BSA, reacting with rabbit antisera, washing, and reacting with radio-iodinated goat anti-rabbit IgG.

XIV Purification of Naturally Occurring HSPDE10A Using Specific Antibodies

Naturally occurring or recombinant HSPDE10A is purified by immunoaffinity chromatography using antibodies specific for HSPDE10A. An immunoaffinity column is constructed by covalently coupling anti-HSPDE10A antibody to CNBr-activated SEPHAROSE resin (APB). After the coupling, the resin is blocked and washed according to the manufacturer's instructions.

Media containing HSPDE10A are passed over the immunoaffinity column, and the column is washed under high ionic strength buffers in the presence of detergent which allow the preferential absorbance of HSPDE10A. The column is eluted using a buffer of pH 2 to pH 3 or a high concentration of a chaotrope, urea or thiocyanate ion, which disrupts antibody/HSPDE10A binding; and HSPDE10A is collected.

XV Antibody Arrays

Protein:protein Interactions

An antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Proteomic Profiles

Antibody arrays can also be used for high-throughput screening of recombinant antibodies. Bacteria containing antibody genes are robotically-picked and gridded at high density (up to about 20,000 different double-spotted clones) on a filter. Up to 15 antigens at a time are used to screen for clones to identify those that express binding antibody fragments. These antibody arrays can also be used to identify proteins which are differentially expressed in samples (de Wildt, supra).

XVI Identification of Molecules which Interact with HSPDE10A

HSPDE10A, or biologically active portions thereof, are labeled with ¹²⁵I Bolton-Hunter reagent (Bolton et al. (1973) Biochem J 133:529-539). Candidate molecules previously arrayed in the wells of a multi-well plate are incubated with the labeled HSPDE10A, washed, and any wells with labeled HSPDE10A complex are assayed. Data obtained using different concentrations of HSPDE10A are used to calculate values for the number, affinity, and association of HSPDE10A with the candidate molecules.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| | Selective for PDE type | IC50 for HSPDE10A1 |
|---|---|---|
| Inhibitor | (IC50) | (μM) |
| IBMX | non-selective (2-50 μM) | 40 |
| Zaprinast | PDE5/6 (0.8/0.2 μM) | 8 |
| Milrinone | PDE3 (1 μM) | >200 |
| Rolipram | PDE4 (2.0 μM) | 160 |

TABLE 2

| Program | Description | Reference | Parameter Threshold |
|---|---|---|---|
| ABI FACTURA | A program that removes vector sequences and masks | Perkin-Elmer Applied Biosystems, | |
| | ambiguous bases in nucleic acid sequences. | Foster City, CA. | |
| ABI/PARACEL FDF | A Fast Data Finder useful in comparing and annotating | Perkin-Elmer Applied Biosystems, | Mismatch <50% |
| | amino acid or nucleic acid sequences. | Foster City, CA; Paracel Inc., Pasadena, CA. | |
| ABI AutoAssembler | A program that assembles nucleic acid sequences. | Perkin-Elmer Applied Biosystems, | |
| | | Foster City, CA. | |
| BLAST | A Basic Local Alignment Search Tool useful in sequence | Altschul, S.F. et al. (1990) J. Mol. Biol. | ESTs: Probability value = 1.0E-8 |
| | similarity search for amino acid and nucleic acid sequences. | 215:403-410; Altschul, S.F. et al. (1997) | or less |
| | BLAST includes five functions: blastp, blastn, blastx, | Nucleic Acids Res. 25:3389-3402. | Full Length sequences: Probability |
| | tblastn, and tblastx. | | value = 1.0E-10 or less |
| FASTA | A Pearson and Lipman algorithm that searches for | Pearson, W.R. and D.J. Lipman (1988) Proc. | ESTs. fasta E value = 1 06E-6 |
| | similarity between a query sequence and a group of | Natl. Acad Sci. 85:2444-2448; Pearson, W.R. | Assembled ESTs: fasta Identity = |
| | sequences of the same type. FASTA comprises as least five | (1990) Methods Enzymol. 183:63-98; and | 95% or greater and Match |
| | functions: fasta, tfasta, fastx, tfastx, and ssearch. | Smith, T.F. and M.S. Waterman (1981) Adv. | length = 200 bases or greater; fastx |
| | | Appl. Math. 2:482-489. | E value = 1.0E-8 or less |
| | | | Full Length sequences fastx |
| | | | score = 100 or greater |
| BLIMPS | A BLocks IMProved Searcher that matches a sequence | Henikoff, S and J.G. Henikoff, Nucl. Acid Res., | Score = 1000 or greater; Ratio of |
| | against those in BLOCKS and PRINTS databases to search | 19:6565-72, 1991. J.G. Henikoff and S. | Score/Strength = 0.75 or larger; |
| | for gene families, sequence homology, and structural | Henikoff(1996) Methods Enzymol. 266:88-105; | and Probability value − 1.0E-3 or |
| | fingerprint regions. | and Attwood, T.K. et al. (1997) J. Chem. Inf. | less |
| | | Comput. Sci. 37:417-424. | |
| PFAM | A Hidden Markov Models-based application useful for | Krogh, A. et al. (1994) J. Mol. Biol., 235:1501- | Score = 10-50 bits, depending on |
| | protein family search. | 1531; Sonnhammer, E.L.L. et al. (1988) | individual protein families |
| | | Nucleic Acids Res. 26:320-322. | |
| ProfileScan | An algorithm that searches for structural and sequence | Gribskov, M. et al. (1988) CABIOS 4:61-66; | Score = 4.0 or greater |
| | motifs in protein sequences that match sequence patterns | Gribskov, et al. (1989) Methods Enzymol. | |
| | defined in Prosite. | 183:146-159; Bairoch, A. et al. (1997) Nucleic | |
| | | Acids Res. 25:217-221. | |
| Phred | A base-calling algorithm that examines automated | Ewing, B. et al. (1998) Genome | |
| | sequencer traces with high sensitivity and probability. | Res. 8:175-185; Ewing, B. and P. | |
| | | Green (1998) Genome Res. 8:186- | |
| | | 194. | |
| Phrap | A Phils Revised Assembly Program including SWAT and | Smith, T.F. and M.S. Waterman (1981) Adv. | Score = 120 or greater; Match |
| | CrossMatch, programs based on efficient implementation of | Appl. Math. 2:482-489; Smith, T.F. and M.S. | length = 56 or greater |
| | the Smith-Waterman algorithm, useful in searching | Waterman (1981) J. Mol. Biol. 147:195-197; | |
| | sequence homology and assembling DNA sequences. | and Green, P., University of Washington, | |
| | | Seattle, WA. | |
| Consed | A graphical tool for viewing and editing Phrap assemblies | Gordon, D. et al. (1998) Genome | |
| | | Res. 8:195-202. | |
| SPScan | A weight matrix analysis program that scans protein | Nielson, H. et al. (1997) Protein Engineering | Score = 5 or greater |
| | sequences for the presence of secretory signal peptides. | 10:1-6; Claverie, J.M. and S. Audic (1997) | |
| | | CABIOS 12:431-439. | |
| Motifs | A program that searches amino acid sequences for patterns | Bairoch et al. supra; Wisconsin | |
| | that matched those defined in Prosite. | Package Program Manual, version | |
| | | 9, page M51-59, Genetics Computer | |
| | | Group, Madison, WI. | |

What is claimed is:

1. A purified protein comprising the amino acid sequence of SEQ ID NO:1, SEQ ID NO:3, or a portion thereof.

2. An antibody that specifically binds the protein of claim 1.

3. The antibody of claim 2, wherein the antibody is selected from a polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein.

4. A method of making a polyclonal antibody that specifically binds a protein, the method comprising:
a) immunizing a animal with a protein having the amino acid sequence of SEQ ID NO:1under conditions to elicit an antibody response;
b) isolating animal antibodies;
c) attaching the protein to a substrate;
d) contacting the substrate with isolated antibodies under conditions to form an antibody:protein complex;
e) dissociating the antibodies from the complex so formed, thereby obtaining polyclonal antibodies with the specificity of the antibody of claim 2.

5. A polyclonal antibody produced by the method of claim 3.

6. A method of preparing a monoclonal antibody that specifically binds a protein, the method comprising:
a) immunizing a animal with a protein having the amino acid sequence of SEQ ID NO:1 under conditions to elicit an antibody response;
b) isolating antibody-producing cells from the animal;
c) fusing the antibody-producing cells with immortalized cells in culture to form monoclonal antibody producing hybridoma cells;
d) culturing the hybridoma cells; and
e) isolating monoclonal antibodies with the specificity of the antibody of claim 2 from culture.

7. A monoclonal antibody produced by the method of claim 6.

8. A method for using an antibody to immunopurify a protein comprising:
a) attaching the antibody of claim 2 to a substrate,
b) exposing the antibody to a sample containing protein under conditions to allow antibody:protein complexes to form,
c) dissociating the protein from the complex, and
d) collecting the purified protein.

9. A method for using an antibody to detect expression of a protein in a sample, the method comprising:
a) combining the antibody of claim 2 with a sample under conditions which allow the formation of antibody:protein complexes; and
b) detecting complex formation, wherein complex formation indicates expression of the protein in the sample.

10. The method of claim 9 wherein the sample is biopsied prostate.

11. The method of claim 9 wherein the complex formation is compared with standards and is diagnostic of adenofibromatous hyperplasia.

12. The method of claim 9, wherein the antibody of claim 2 is attached to a substrate.

13. A composition comprising an antibody of claim 2 and a labeling moiety.

14. A composition comprising an antibody of claim 2 and a pharmaceutical agent.

15. A method for treating adenofibromatous hyperplasia, the method comprising administering the antibody of claim 2 to a subject in need of such treatment.

16. A method for treating a cancer, the method comprising administering the antibody of claim 2 to a subject in need of such treatment.

17. A method for treating an immune disorder, the method comprising administering the antibody of claim 2 to a subject in need of such treatment.

18. A purified agonist that specifically binds the protein of claim 1.

19. A purified antagonist that specifically binds the protein of claim 1.

20. A method for using an antibody that specifically binds the protein to evaluate treatment of adenofibromatous hyperplasia of the prostate comprising contacting the antibody of claim 2 with a sample from a patient, detecting complex formation between the antibody and protein, comparing complex formation with standards, wherein the difference in complex formation indicates efficacy of treatment.


SEQUENCE LISTING (7 sequences)

SEQ ID NO: 1 - protein, 490 aa, Homo sapiens
  misc_feature: Incyte ID No HSPDE10A1
MSPKCSADAENSFKESMEKSSYSDWLINNSIAELVASTGLPVNISDAYQDPRFDAEADQISGFHIRSVLCVPIWNSNHQIIGVAQVLNRLDGKPFDDADQRLFEAFVIFCGLGINNTIMYDQVKKSWAKQSVALDVLSYHATCSKAEVDKFKAANIPLVSELAIDDIHFDDFSLDVDAMITAALRMFMELGMVQKFKIDYETLCRWLLTVRKNYRMVLYHNWRHAFNVCQLMFAMLTTAGFQDILTEVEILAVIVGCLCHDLDHRGTNNAFQAKSGSALAQLYGTSATLEHHHFNHAVMILQSEGHNIFANLSSKEYSDLMQLLKQSILATDLTLYFERRTEFFELVSKGEYDWNIKNHRDIFRSMLMTACDLGAVTKPWEISRQVAELVTSEFFEQGDRERLELKLTPSAIFDRNRKDELPRLQLEWIDSICMPLYQALVKVNVKLKPMLDSVATNRSKWEELHQKRLLASTASSSSPASVMVAKEDRN

SEQ ID NO: 2 - DNA, 1784 bp, Homo sapiens
  misc_feature: Incyte ID No HSPDE10A1
tggaaagatgttacttcatctcccaggtttgctcactgcaaatacaatcctgagaactgaactagggccttaaagtcctgacatgcatggcttggttttgtggattgcctctctcaacaggtggtgaaatttaccaaatcctttgaattgatgtccccaaagtgcagtgctgatgctgagaacagtttcaaagaaagcatggagaaatcatcatactccgactggctaataaataacagcattgctgagctggttgcttcaacaggccttccagtgaacatcagtgatgcctaccaggatccgcgctttgatgcagaggcagaccagatatctggttttcacataagatctgttctttgtgtccctatttggaatagcaaccaccaaataattggagtggctcaagtgttaaacagacttgatgggaaaccttttgatgatgcagatcaacgactttttgaggcttttgtcatcttttgtggacttggcatcaacaacacaattatgtatgatcaagtgaagaagtcctgggccaagcagtctgtggctcttgatgtgctatcataccatgcaacatgttcaaaagctgaagttgacaagtttaaggcagccaacatccctctggtgtcagaacttgccatcgatgacattcattttgatgacttttctctcgacgttgatgccatgatcacagctgctctccggatgttcatggagctggggatggtacagaaatttaaaattgactatgagacactgtgtaggtggcttttgacagtgaggaaaaactatcggatggttctataccacaactggagacatgccttcaacgtgtgtcagctgatgttcgcgatgttaaccactgctgggtttcaagacattctgaccgaggtggaaattttagcggtgattgtgggatgcctgtgtcatgacctcgaccacaggggaaccaacaatgccttccaagctaagagtggctctgccctggcccaactctatggaacctctgctaccttggagcatcaccatttcaaccacgccgtgatgatccttcaaagtgagggtcacaatatctttgctaacctgtcctccaaggaatatagtgaccttatgcagcttttgaagcagtcaatattggcaacagacctcacgctgtactttgagaggagaactgaattctttgaacttgtcagtaaaggagaatacgattggaacatcaaaaaccatcgtgatatatttcgatcaatgttaatgacagcctgtgaccttggagccgtgaccaaaccgtgggagatctccagacaggtggcagaacttgtaaccagtgagttcttcgaacaaggagatcgggagagattagagctcaaactcactccttcagcaatttttgatcggaaccggaaggatgaactgcctcggttgcaactggagtggattgatagcatctgcatgcctttgtatcaggcactggtgaaggtcaacgtgaaactgaagccgatgctagattcagtagctacaaacagaagtaagtgggaagagctacaccaaaaacgactgctggcctcaactgcctcatcctcctcccctgccagtgttatggtagccaaggaagacaggaactaaacctccaggtcagctgcagctgcaaaatgactacagcctgaagggccattttcagtccagcaatgtcatccttttgttcttttagctcagaaagacctaacatctcaaggatgcactgggaaccatgcctgggctttcaccttgaagcatggtcagcagca

SEQ ID NO: 3 - protein, 367 aa, Homo sapiens
  misc_feature: Incyte ID No HSPDE10A2
MSPKCSADAENSFKESMEKSSYSDWLINNSIAELVASTGLPVNISDAYQDPRFDAEADQISGFHIRSVLCVPIWNSNHQIIGVAQVLNRLDGKPFDDADQRLFEAFVIFCGLGINNTIMYDQVKKSWAKQSVALDVLSYHATCSKAEVDKFKAANIPLVSELAIDDIHFDDFSLDVDAMITAALRMFMELGMVQKFKIDYETLCRWLLTVRKNYRMVLYHNWRHAFNVCQLMFAMLTTAGFQDILTEVEILAVIVGCLCHDLDHRGTNNAFQAKSGSALAQLYGTSATLEHHHFNHAVMILQSEGHNIFANLSSKEYSDLMQLLKQSILATDLTLYFEEKVRNTSPGAVNHLPGTSNLQLFFGAPPY

SEQ ID NO: 4 - DNA, 1982 bp, Homo sapiens
  misc_feature: Incyte ID No HSPDE10A2
tcgacgtggaaagatgttacttcatctcccaggtttgctcactgcaaatacaatcctgagaactgaactagggccttaaagtcctgacatgcatggcttggttttgtggattgcctctctcaacaggtggtgaaatttaccaaatcctttgaattgatgtccccaaagtgcagtgctgatgctgagaacagtttcaaagaaagcatggagaaatcatcatactccgactggctaataaataacagcattgctgagctggttgcttcaacaggccttccagtgaacatcagtgatgcctaccaggatccgcgctttgatgcagaggcagaccagatatctggttttcacataagatctgttctttgtgtccctatttggaatagcaaccaccaaataattggagtggctcaagtgttaaacagacttgatgggaaaccttttgatgatgcagatcaacgactttttgaggcttttgtcatcttttgtggacttggcatcaacaacacaattatgtatgatcaagtgaagaagtcctgggccaagcagtctgtggctcttgatgtgctatcataccatgcaacatgttcaaaagctgaagttgacaagtttaaggcagccaacatccctctggtgtcagaacttgccatcgatgacattcattttgatgacttttctctcgacgttgatgccatgatcacagctgctctccggatgttcatggagctggggatggtacagaaatttaaaattgactatgagacactgtgtaggtggcttttgacagtgaggaaaaactatcggatggttctataccacaactggagacatgccttcaacgtgtgtcagctgatgttcgcgatgttaaccactgctgggtttcaagacattctgaccgaggtggaaattttagcggtgattgtgggatgcctgtgtcatgacctcgaccacaggggaaccaacaatgccttccaagctaagagtggctctgccctggcccaactctatggaacctctgctaccttggagcatcaccatttcaaccacgccgtgatgatccttcaaagtgagggtcacaatatctttgctaacctgtcctccaaggaatatagtgaccttatgcagcttttgaagcagtcaatattggcaacagacctcacgctgtactttgaggagaaggtcagaaatacatcacctggagctgtgaaccacctacctggcacaagcaatctgcagctcttctttggagcacccccttattgatgatggaaagaaccctgtctgtgtctgccttgatacttggtattgccttggtacagcagcctgtgatgctgttacatagcatgagggctgctggccccactgtccatacacttacaacatgaaaagctatctggcccaaaggtttatgctacacatagtttacaaagattatctcagagggcagaaccgggaggctggggacttataatctacccagaaggaaaagttcttccttatagaagatttcaattaacacacatggaaaggtggaaatggaaaaatcatcagctggcaaataccacggtagtaatttttattggcaacaataaatctttctgtaactgccctgggaccttgaacaagtcacttcaccttccttcaccttgagtttcctcacctataaaatgagagaattaataggagatttttctcaaaagttccatacagccctaccagtctataactataatgaaaattcaaacatagaaaagaagtcattctatgacccagcaattttacatatacatgtacatattcatatacacagagagagagaactcacacaaattcacaaggaaacatgtacaaggtggttcatagctgcattgtatgtaatagcaagaaatattagaaaaatataaattttcatcttccaggaaatgggtaaatagacagtggtataataatagatggaaatagcatacatcagtatgaaggaatgg

SEQ ID NO: 5 - protein, 875 aa, Homo sapiens
  misc_feature: GenBank ID No g3355606
MERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 6 - DNA, 43 bp, Homo sapiens
  misc_feature: Incyte ID No UNKNOWN
ccaaatcccggtccgagatgtccccaaagtgcagtgctgatgc

SEQ ID NO: 7 - DNA, 41 bp, Homo sapiens
  misc_feature: Incyte ID No UNKNOWN
cgggtacctcgagttattagttcctgtcttccttggctacc